(19) United States
(12) Patent Application Publication
(10) Pub. No.: US 2015/0132229 A1
(43) Pub. Date: May 14, 2015

(54) BIS AZAINOSITOL HEAVY METAL COMPLEXES FOR X-RAY IMAGING

(71) Applicant: BAYER PHARMA AKTIENGESELLSCHAFT, Berlin (DE)

(72) Inventors: Markus Berger, Berlin (DE); Heribert Schmitt-Willich, Berlin (DE); Detlev Sülzle, Berlin (DE); Hubertus Pietsch, Kleinmachnow (DE); Thomas Frenzel, Berlin (DE); Gregor Jost, Berlin (DE); Kaspar Hegetschweiler, St. Ingbert (DE); Christian Neis, Hamburg (DE); Silvia Lauria, Saarbrucken (DE)

(21) Appl. No.: 14/402,050

(22) PCT Filed: Apr. 25, 2013
(86) PCT No.: PCT/EP2013/058590

§ 371 Date: Nov. 18, 2014

(30) Foreign Application Priority Data
May 18, 2012 (EP) 12075048.4

(51) Int. Cl. C07F 7/00; C07F 5/00; A61K 49/04

(52) U.S. Cl. CPC C07F 7/003; A61K 49/04; C07F 5/003
USPC 424/9.42; 556/55; 534/16

(57) ABSTRACT

The present invention describes a new class of trinuclear heavy metal complexes comprising two hexadentate azainositol tricarboxylic acid ligands, a method for their preparation and their use as X-ray contrast agents.

The present invention describes a new class of bis azainositol heavy metal complexes, especially trinuclear heavy metal complexes comprising two hexadentate azainositol tricarboxylic acid ligands, a method for their preparation and their use as X-ray contrast agents.

### BACKGROUND OF THE INVENTION

The synthesis and co-ordination chemistry of 1,3,5-triamino-1,3,5-trideoxy-cis-inositol (taci) and a multitude of derivatives of this cyclohexane-based polyamino-polyalcohol have widely been examined in the past by Hegetschweiler et al. (Chem. Soc. Rev. 1999, 28, 239). Among other things, the ability of taci and of the hexa-N,N′,N″-methylated ligand tdci to form trinuclear complexes of the composition [M₃(H₋₃taci)₂]³⁺ and [M₃(H₋₃tdci)₂]³⁺, respectively, with a unique, sandwich-type cage structure in the presence of heavy metals M^{III} like Bi^{III} or a series of lanthanides was described (Chem. Soc. Rev. 1999, 28, 239; Inorg. Chem. 1993, 32, 2699; Inorg. Chem. 1998, 37, 6698). But, due to their moderate solubility in water and their deficient thermodynamic stability, these complexes proved not to be suitable for in vivo applications. The efficacy of complexation can directly be deduced from the thermodynamic stability constant log K (K=[ML]×[M]⁻¹×[L]⁻¹) of the metal complex which, taking the basicity of the ligand into account, allows to calculate the free metal concentration (pM=−log [M]_{free}) under defined conditions ([M]ₜₒₜ=10⁻⁶ mol/l; [L]ₜₒₜ=10⁻⁵ mol/l; pH=7.4). Besides the high thermodynamic stability a high kinetic stability can additionally avoid the dissociation of metal complexes and thereby improve the in vivo safety. Chapon et al. (J. All. Comp. 2001, 323-324, 128) determined the stability constants for lanthanide complexes with taci in aqueous solution. The corresponding pM values that reflect the complex stability at physiological pH of 7.4 vary in the range from 6.3 (for Eu³⁺) to 8.6 (for Lu³⁺) which is insufficient in view of the required in vivo safety (vide supra, section 3).

Complex formation of taci with more than 30 metal ions has been investigated and the metal cations can be divided into five categories according to the adopted coordination mode that was verified by crystal structure analyses (Chem. Soc. Rev. 1999, 28, 239). Although this classification helpfully reviews the coordination properties of taci, it has to be pointed out that multiple metals do not fit into the presented scheme. As a consequence, a prediction of the preferred coordination mode for metals that have not been described so far is often ambiguous. In addition to that, it was demonstrated that modifications at the ligand backbone can have a strong impact on the coordination behavior (Inorg. Chem. 1997, 36, 4121). This is not only reflected in the structural characteristics of the metal complexes but can often lead to unpredictable changes in their thermodynamic and/or kinetic complex stability, water solubility and other physicochemical parameters. The ability to form trinuclear heavy metal complexes with a sandwich-type cage structure was neither reported before for the propionate nor the acetate derivatives of taci nor for any other derivative in which additional coordinating groups are attached to the taci backbone.

Moreover, the synthesis of mononuclear carboxylic acid derived taci metal complexes has been reported by Laboratorien Hausmann AG, St. Gallen, CH in DE 40 28 139 A1 and WO 92/04056 A1 for iron, gadolinium. A possible application of its mononuclear, radioactive metal complexes as radiopharmaceuticals was also claimed.

All-cis-1,3,5-triamino-2,4,6-cyclohexane triol derivatives, their use and methods for their preparation were also described by Laboratorien Hausmann AG in EP, A, 190 676.

Byk Gulden Lomberg Chemische Fabrik GmbH described taci based transition metal complexes for magnetic resonance diagnostics in WO 91/10454.

Nycomed AS in WO 90/08138 described heterocyclic chelating agents for the preparation of diagnostic and therapeutic agents for magnetic resonance imaging, scintigraphy, ultrasound imaging, radiotherapy and heavy metal detoxification.

The formation of trinuclear iron^{III} complexes was suggested by G. Welti (Dissertation, Zürich 1998) for an acetate and by A. Egli (Dissertation, Zürich 1994) for a 2-hydroxybenzyl derivative of taci. G. Welti also described the synthesis of Rhenium^{V} and Rhenium^{VII} complexes of acetate derived ligands based on taci with a M₁L₁ stoichiometry.

D. P. Taylor & G. R. Choppin (Inorg. Chim. Acta 2007, 360, 3712) described the formation of mononuclear complexes with lanthanides with similar derived ligands and determined the thermodynamic stability for complexes with Eu³⁺ with a pM value of 6.0 even lower than Eu³⁺ complexes of unmodified taci.

Since the iodine content of iodinated CT contrast agents that are administrated today is 45% or even higher, polynuclear metal complexes are needed to significantly improve the attenuation properties. Mononuclear metal complexes like (NMG)₂GdDTPA (Janon E. A. Am. J. Roentgen 1989, 152, 1348) or YbDTPA (Unger E., Gutierrez F. Invest. Radiol. 1986, 21, 802) proved to be well-tolerated alternatives for patients that are contraindicated for iodinated agents but a reduction in the radiation doses and/or the contrast agent dosages can only be achieved when the metal content is comparable to the content of iodine in the current X-ray contrast agents. All compounds described above in or out of the context with diagnostic applications hold either only one metal center bound to the complex and the metal content of ≦30% is significantly lower than 40% or the present metal is, not suited for a X-ray CT application due to its low absorption coefficient, i.e. iron.

Hafnium and lanthanides are characterized by a higher absorption coefficient for X-rays than iodine, especially in the range of tube voltages normally used in modern CT. A modern CT X-ray-tube, however, requires a minimum voltage of about 70 kV and reaches maximum voltage of 160 kV. As future technical developments in CT will not substantially change these parameters, iodine generally does not provide ideal attenuation features for this technology. In comparison to iodine the attenuation optimum (k-edge) of hafnium and lanthanides corresponds better to the ranges of voltages used in CT. Therefore the new hafnium and lanthanides complexes require a similar or lower contrast media dosage than conventional trisiodinated contrast agents.

The use of hafnium and lanthanides based contrast agents will allow more flexibility for CT scanning protocols and lead to scan protocols that provide equivalent diagnostic value at lower radiation doses. Especially this feature is of high importance for CT. As technical development goals in terms of spatial and temporal resolution have approached the limit of clinical significance, reduction of the radiation burden of CT scanning has today become a central aspect of the development of new CT scanners and X-ray machines. Following the widely accepted ALARA-rule (radiation exposure has to be reduced to levels: As Low As Reasonably Achievable), the new hafnium and lanthanides based contrast agents will contribute to high-quality diagnostic imaging at reduced radiation exposure.

In summary, the state of the art described above consists of either physiologically stable heavy metal complexes with a low metal content per molecule or complexes with a high metal content, which are not thermodynamically stable enough for a physiological application or hold a metal that is not suitable for a diagnostic X-ray CT application.

The aim of the present invention was to provide sufficiently stable, water soluble and well tolerated hafnium and lanthanide complexes with a higher metal content for use as X-ray contrast agents in diagnostic imaging, especially in modern computed tomography.

This aim was achieved by the provision of the compounds of the present invention. It has now been found, that tri-N,N′,N″-carboxylic acid derivatives of taci (L) effectively form new complexes with lanthanides and hafnium of a M₃L₂ stoichiometry which grants a high metal content of >35% for the compounds of the present invention. Surprisingly, it was observed that the complexes described in this patent application show a very high stability in aqueous solution for this type of stoichiometry under heat sterilization conditions and have an excellent tolerability in experimental animals as well as a high in vivo stability.

After intravenous injection the compounds of the present invention are excreted fast and quantitatively via the kidneys, comparable to the well established trisiodinated X-ray contrast agents.

The invention of suitable new bis-azainositol heavy metal complexes enables for the first time the practical use of this compound class as X-ray contrast agents in diagnostic imaging.

By enabling and developing new novel hafnium-based and lanthanides-based contrast agents a clear advantage over the existing iodine-based contrast agents is offered as the radiative dose for the higher absorption coefficient of hafnium-based and lanthanides-based contrast agents is significantly reduced in comparison to the iodine-based contrast agents.

### DETAILED DESCRIPTION OF THE INVENTION

In a first aspect, the present invention is directed to bis azainositol heavy metal complexes, especially trinuclear heavy metal complexes comprising two hexadentate azainositol tricarboxylic acid ligands.

In a second aspect, the invention is directed to compounds of the general formula (I),

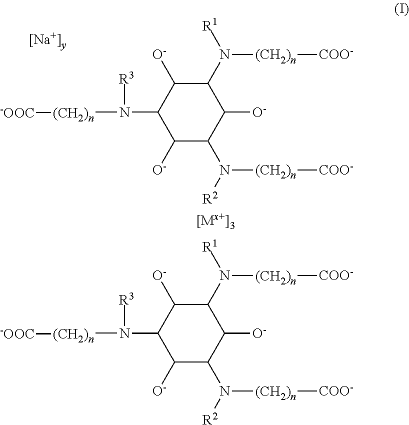

wherein
the substituents at the cyclo hexyl ring exhibit an all-cis configuration;
M is Lanthanum, Cerium, Praseodymium, Neodymium, Samarium, Europium, Gadolinium, Terbium, Dysprosium, Holmium, Erbium, Thulium, Ytterbium, Lutetium, Hafnium or Bismuth;
R¹, R² and R³ are independently selected from H or methyl;
n is 1 or 2;
x is 3 or 4;
and
y is 0 or 3;
   with the proviso that (3 times x)+y is 12;
      including any protonated species and any deprotonated species of said compounds, including all isomeric forms of said compounds, including but not limited to enantiomers, diastereomers, regioisomers and mixtures thereof, and any pharmaceutically acceptable salt of such compounds or hydrates thereof.

In a preferred embodiment, the invention relates to compounds of formula (I), supra, wherein M is Gadolinium, Terbium, Dysprosium, Holmium, Erbium, Thulium, Ytterbium, Lutetium, Hafnium or Bismuth.

In a specially preferred embodiment, the invention relates to compounds of formula (I), supra, wherein M is Hafnium (Hf).

In another preferred embodiment, the invention relates to compounds of formula (I), supra, wherein R¹, R² and R³ are methyl.

It is to be understood that the present invention relates also to any combination of the preferred embodiments described above.

In another specially preferred embodiment, the invention relates to compounds of formula (I), supra, wherein M is Hafnium (Hf), and R¹, R² and R³ are methyl.

Trinuclear complexes of the general formula (I), which are charged at physiological pH, can be neutralized by addition of suitable, physiologically biocompatible counter ions, e.g. sodium ions or suitable cations of organic bases including, among others, those of primary, secondary or tertiary amines, for example N-methylglucamine. Lysine, arginine or ornithine are suitable cations of amino acids, as generally are those of other basic naturally occurring amino acids.

A preferred compound of the general formula (I) is [Hf₃(H₋₃tacita)₂]=Bis{μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}trihafnium(IV)

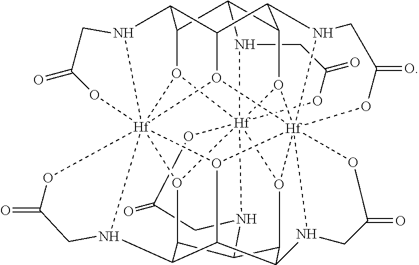

Another preferred compound of the general formula (I) is Na₃[Lu₃(H₋₃tacita)₂]=Trisodium bis{μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}trilutetate(III)

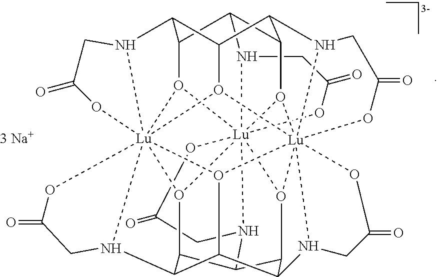

Another preferred compound of the general formula (I) is Na₃[Gd₃(H₋₃tacita)₂]=Trisodium bis{μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}trigadolinate(III)

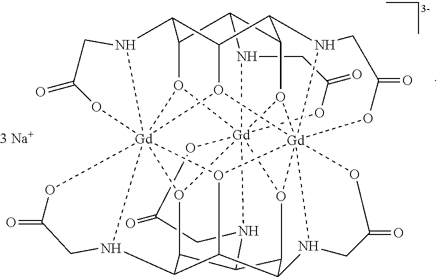

Another preferred compound of the general formula (I) is Na₃[Ho₃(H₋₃tacita)₂]=Trisodium bis{μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}triholmate(III)

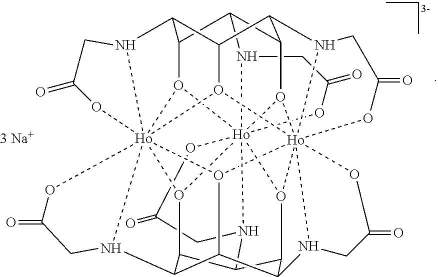

Another preferred compound of the general formula (I) is Na₃[Er₃(H₋₃tacita)₂]=Trisodium bis{μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}trierbate(III)

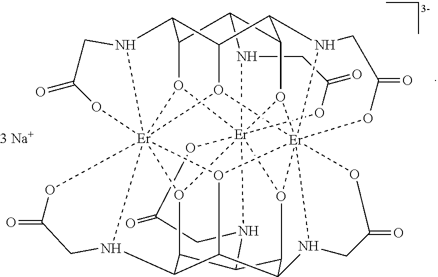

Another preferred compound of the general formula (I) is Na₃[Yb₃(H₋₃tacita)₂]=Trisodium bis{μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}triytterbate(III)

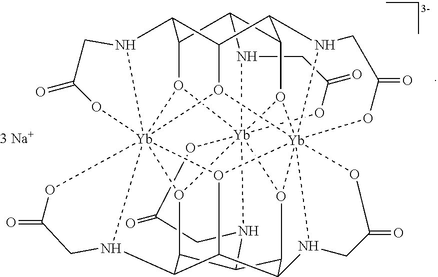

Another preferred compound of the general formula (I) is [Hf₃(H₋₃macita)₂]=Bis{μ₃-[(all-cis)-2-{[(carboxy-1κO)methyl](methyl)amino-1κN}-4-{[(carboxy-2κO)methyl](methyl)amino-2κN}-6-{[(carboxy-3κO)methyl](methyl)amino-3κN}cyclohexane-1,3,5-triolate-1κ²O¹,O³:2κ²O³,O⁵:3κ²O¹,O⁵]}trihafnium(IV)

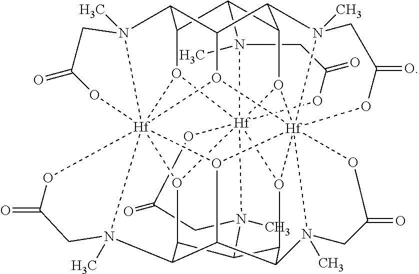

Another preferred compound of the general formula (I) is Na₃[Lu₃(H₋₃macita)₂]=Trisodium bis{μ₃-[(all-cis)-2-{[(carboxy-1κO)methyl](methyl)-amino-1κN}-4-{[(carboxy-2κO)methyl](methyl)amino-2κN}-6-{[(carboxy-3κO)methyl]-(methyl)amino-—3κN}cyclohexane-1,3,5-triolate-1κ²O¹,O³:2κ²O³,O⁵:3κ²O¹,O⁵]}trilutetate(III)

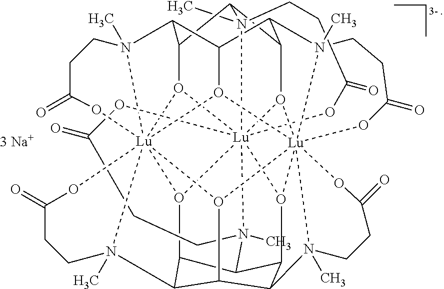

Another preferred compound of the general formula (I) is Na₃[Gd₃(H₋₃macita)₂]=Trisodium bis{μ₃-[(all-cis)-2-{[(carboxy-1κO)methyl](methyl)-amino-1κN}-4-{[(carboxy-2κO)methyl](methyl)amino-2κN}-6-{[(Carboxy-3κO)methyl](methyl)amino-3κN}cyclohexane-1,3,5-triolate-1κ²O¹,O³:2κ²O³,O⁵:3κ²O¹,O⁵]}trigadolinate(II)

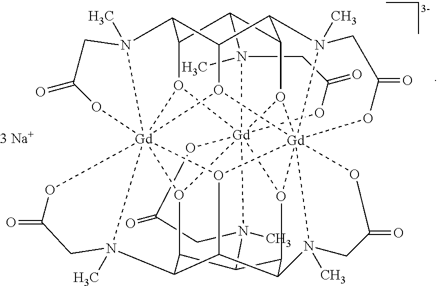

Another preferred compound of the general formula (I) is Na₃[Ho₃(H₋₃macita)₂]=Trisodium bis{μ₃-[(all-cis)-2-{[(carboxy-1κO)methyl](methyl)-amino-1κN}-4-{[(carboxy-2κO)methyl](methyl)amino-2κN}-6-{[(carboxy-3κO)methyl]-(methyl)amino-3κN}cyclohexane-1,3,5-triolate-1κ²O¹,O³:2κ²O³,O⁵:3κ²O¹,O⁵]}triholmate (III)

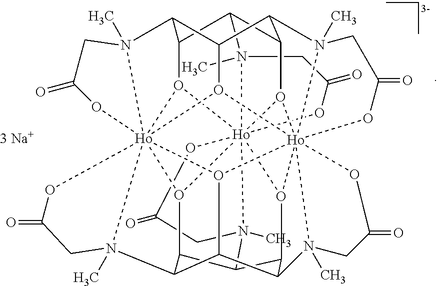

Another preferred compound of the general formula (I) is Na₃[Er₃(H₋₃macita)₂]=Trisodium bis{μ₃-[(all-cis)-2-{[(carboxy-1κO)methyl](methyl)-amino-1κN}-4-{[(carboxy-2κO)methyl](methyl)amino-2κN}-6-{[(carboxy-3κO)methyl](methyl)amino-3κN}cyclohexane-1,3,5-triolate-1κ²O¹,O³:2κ²O³,O⁵:3κ²O¹,O⁵]}trierbate(III)

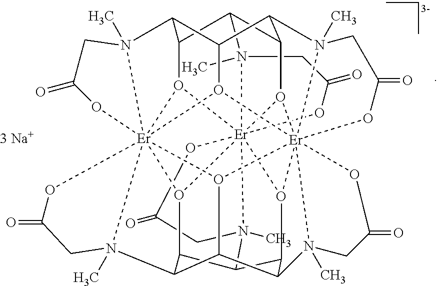

Another preferred compound of the general formula (I) is Na₃[Yb₃(H₋₃macita)₂]=Trisodium bis{μ₃-[(all-cis)-2-{[(carboxy-1κO)methyl](methyl)-amino-1κN}-4-{[(carboxy-2κO)methyl](methyl)amino-2κN}-6-{[(carboxy-3κO)methyl](methyl)amino-3κN}cyclohexane-1,3,5-triolate-1κ²O¹,O³:2κ²O³,O⁵:3κ²O¹,O⁵]}triytterbate(III)

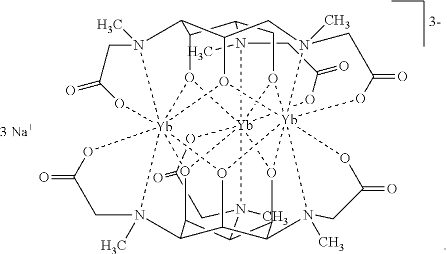

Another preferred compound of the general formula (I) is [Hf₃(H₋₃tacitp)₂]=Bis{μ₃-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}trihafnium(IV)

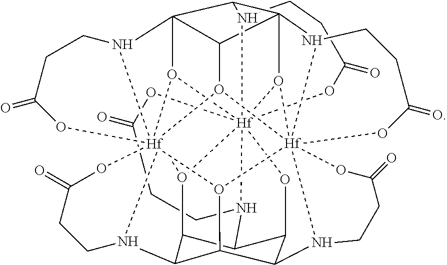

Another preferred compound of the general formula (I) is Na₃[Lu₃(H₋₃tacitp)₂]=Trisodium bis{μ₃-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}trilutetate(III)

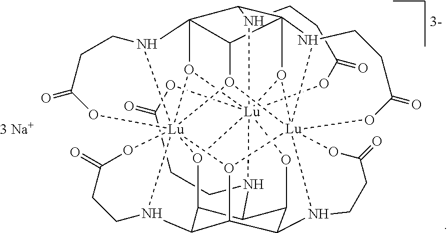

Another preferred compound of the general formula (I) is Na₃[Ho₃(H₋₃tacitp)₂]=Trisodium bis{μ₃-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}triholmate(III)

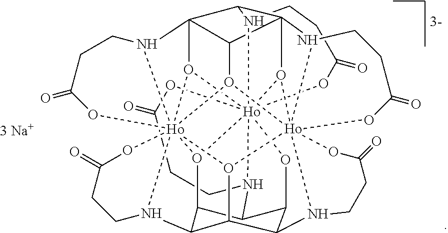

Another preferred compound of the general formula (I) is Na₃[Er₃(H₋₃tacitp)₂]=Trisodium bis{μ₃-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}trierbate(III)

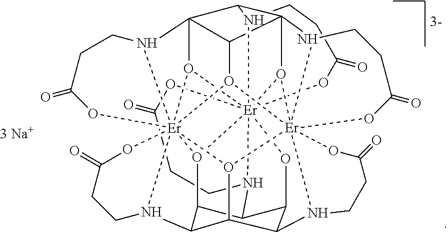

Another preferred compound of the general formula (I) is Na₃[Yb₃(H₋₃tacitp)₂]=Trisodium bis{μ₃-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}triytterbate(III)

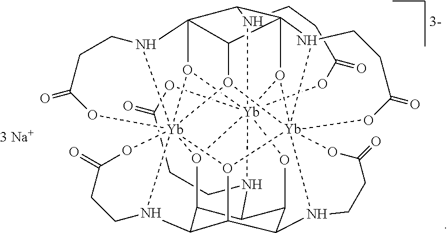

Another preferred compound of the general formula (I) is [Hf₃(H₋₃macitp)₂]=Bis{μ₃-[(all-cis)-2-{[(carboxy-1κO)ethyl](methyl)amino-1κN}-4-{[(carboxy-2κO)ethyl](methyl)amino-2κN}-6-{[(carboxy-3κO)ethyl](methyl)amino-3κN}cyclohexane-1,3,5-triolate-1κ²O¹,O³:2κ²O³,O⁵:3κ²O¹,O⁵]}trihafnium(IV)

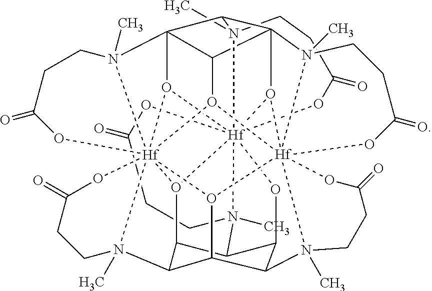

Another preferred compound of the general formula (I) is Na₃[Lu₃(H₋₃macitp)₂]=Trisodium bis{μ₃-[(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}trilutetate(III)

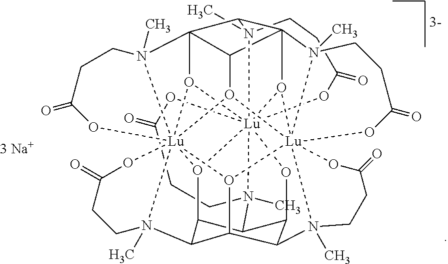

Another preferred compound of the general formula (I) is Na₃[Gd₃(H₋₃macitp)₂]=Trisodium bis{N₃-[(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}trigadolinate(III)

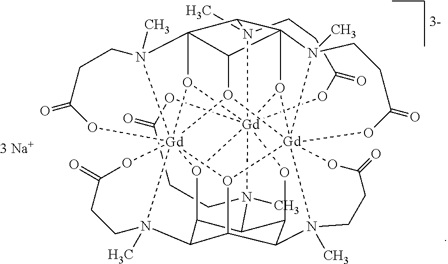

Another preferred compound of the general formula (I) is Na₃[Ho₃(H₋₃macitp)₂]=Trisodium bis{μ₃-[(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}triholmate(III)

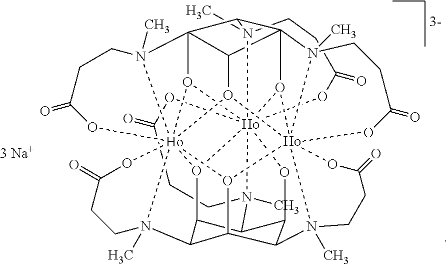

Another preferred compound of the general formula (I) is Na₃[Er₃(H₋₃macitp)₂]=Trisodium bis{μ₃-[(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}trierbate(III)

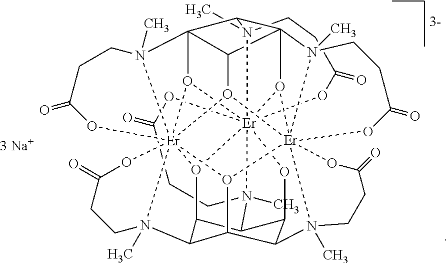

Another preferred compound of the general formula (I) is Na₃[Yb₃(H₋₃macitp)₂=Trisodium bis{μ₃-(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}triytterbate(II)

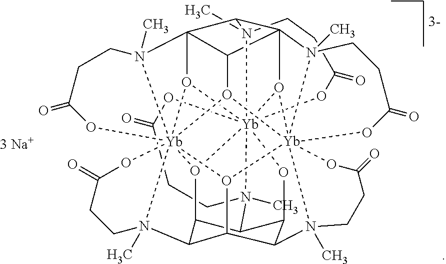

In a third aspect, the invention is directed to the process for the preparation of the compounds of the general formula (I).

In a fourth aspect, the invention is directed to the process for the preparation of the compounds of the general formula (I) from carboxylic acids of the general formula (II),

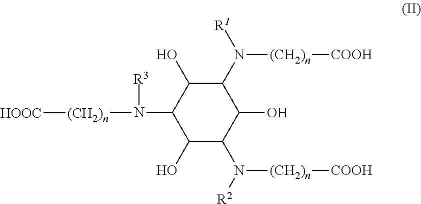

wherein
the substituents at the cyclo hexyl ring exhibit an all-cis configuration;
R¹, R² and R³ are independently H or methyl;
and
n is 1 or 2;
   and metal halogenides,
   wherein
metal is Lanthanum, Cerium, Praseodymium, Neodymium, Samarium, Europium, Gadolinium, Terbium, Dysprosium, Holmium, Erbium, Thulium, Ytterbium, Lutetium, Hafnium or Bismuth;
and
halogenide is either chloride or bromide,
and hydrates thereof,
   in aqueous solution under elevated temperatures ranging from 80° C. to 160° C. in a pH range of 1 to 6 preferably at 90° to 130° C. in a pH range of 2 to 5.

In a fifth aspect, the invention is directed to compounds of general formula (I) for the manufacture of diagnostic agents, especially of X-ray diagnostic agents for administration to humans or animals.

For the manufacture of diagnostic agents, for example the administration to human or animal subjects, the compounds of general formula (I) will conveniently be formulated together with pharmaceutical carriers or excipient. The contrast media of the invention may conveniently contain pharmaceutical formulation aids, for example stabilizers, antioxidants, pH adjusting agents, flavors, and the like. They may be formulated for parenteral or enteral administration or for direct administration into body cavities. For example, parenteral formulations contain a sterile solution or suspension in a concentration range from 150 to 600 mg metal/mL, especially 200 to 450 mg metal/mL of the new azainositol heavy metal complexes according to this invention. Thus the media of the invention may be in conventional pharmaceutical formulations such as solutions, suspensions, dispersions, syrups, etc. in physiologically acceptable carrier media, preferably in water for injections. When the contrast medium is formulated for parenteral administration, it will be preferably isotonic or hypertonic and close to pH 7.4.

Pharmaceutically acceptable salts of the compounds according to the invention also include salts of customary bases, such as, by way of example and by way of preference, alkali metal salts (for example sodium salts), alkaline earth metal salts (for example calcium salts) and ammonium salts, derived from ammonia or organic amines having 1 to 16 carbon atoms, such as, by way of example and by way of preference, N-methylglucamine.

For use as X-ray contrast agent, the media of the invention should generally have a sufficiently high percentage of hafnium or late lanthanide, in particular a contrast medium with a high content of heavy metal per molecule.

### General Synthesis of Compounds of the Invention

The present invention provides carboxylic acid derived ligands based on 1,3,5-triamino-1,3,5-trideoxy-cis-inositol (taci) that can readily form trinuclear, highly stable metal complexes with lanthanides and hafnium useful as X-ray contrast agents. Particularly, the tri-N,N′,N″-acetic acid derivative (tacita) and the tri-N,N′,N″-propionic acid derivative (tacitp) as well as their tri-N,N′,N″-methylated analogs (macita and macitp) were prepared (Scheme 1 & 2).

The ligand tacita was synthesized according to G. Welti (Dissertation, Zürich 1998) using the tri-O-benzylated taci derivative tbca as starting material which was alkylated in the reaction with the sterically demanding agents N,N-diisopropylethylamine and tert-butyl-bromoacetate (Scheme 1). The protecting groups were removed in boiling 6 M hydrochloric acid and pure H₃tacita was isolated by precipitation of the zwitterionic ligand at pH 5.5.

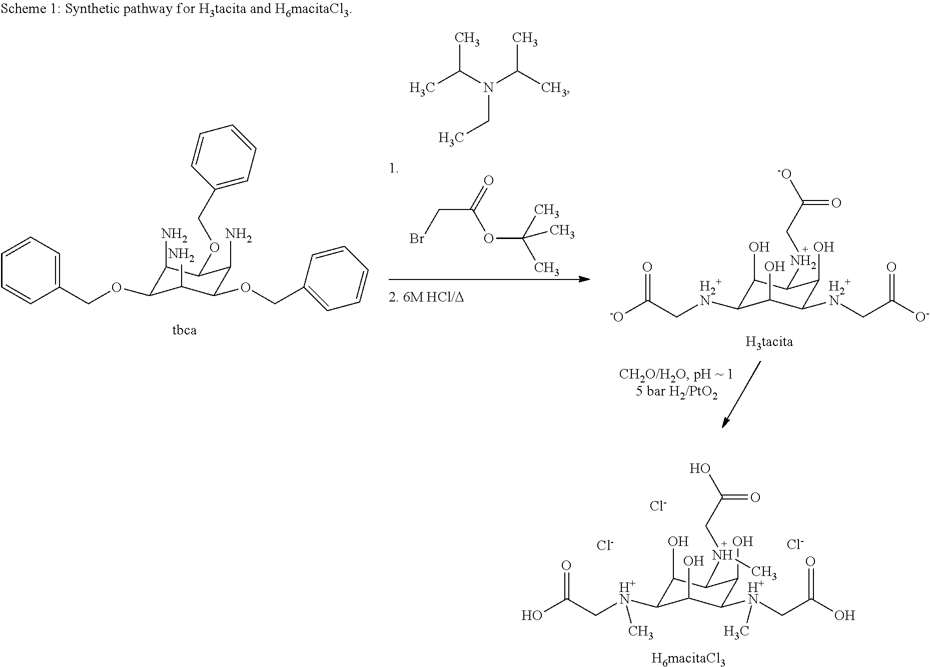

The synthesis of the tri-N,N′,N″-propionic acid derivative (tacitp) was first of all reported by Laboratorien Hausmann AG, St. Gallen, CH, in DE 40 28 139 A1, 1992. Herein, we describe a modified procedure in which the ligand taci dissolved in methanol reacts with acrylonitrile in a first step (Scheme 2). The intermediate was finally hydrolyzed to the tricarboxylic acid in alkaline solution (25% sodium hydroxide). The pure ligand was conveniently obtained in the hydrochloride form by cation exchange chromatography.

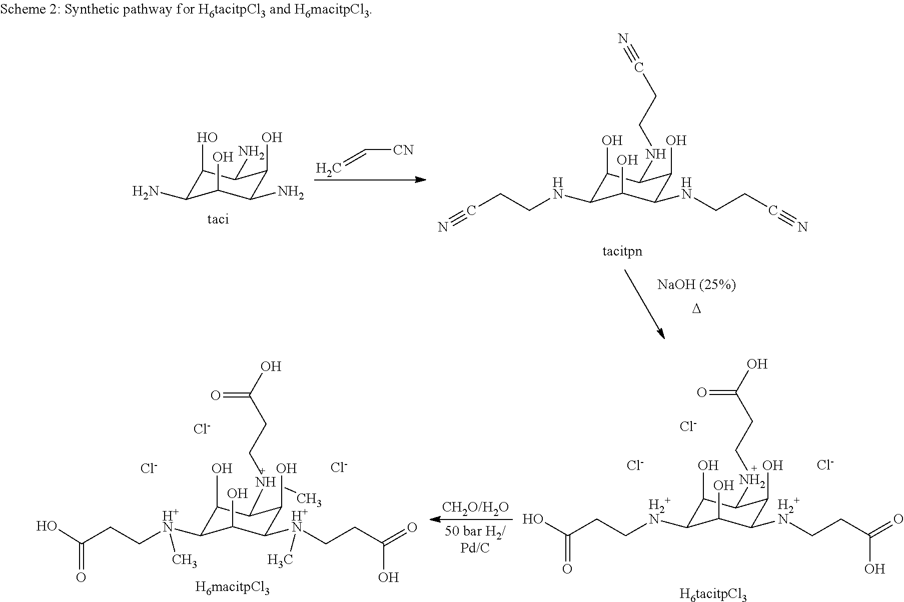

Introduction of additional methyl groups was obtained for tacita as well as for tacitp by catalytic hydrogenation of aqueous solutions of the ligands in the presence of formaldehyde. The ligands were eventually purified and isolated in their hydrochloride form by cation exchange chromatography.

New trinuclear heavy metal complexes of the aforementioned ligands with lanthanides and hafnium were synthesized by adding stoichiometric amounts of a corresponding metal salt to aqueous or methanolic solutions of the ligands (Scheme 3). The reaction mixtures were heated under alkaline (pH 8-9/1-2 h for lanthanide complexes) or acidic conditions (pH 2-3/20 h-3 d for hafnium complexes). Isolation and purification of the desired complexes was obtained by conventional ion exchange chromatography, extraction, precipitation or ultrafiltration methods. Generally, the complexes were characterized by means of elemental analysis (C, H, N), mass spectrometry (ESI-MS) and IR spectroscopy. In addition to that, a metal analysis was performed by ICP-OES for selected compounds. The diamagnetic complexes with Lu³⁺ and Hf⁴⁺ were furthermore examined by NMR spectroscopy revealing in each case the formation of two diastereomeric forms of the trinuclear complexes [M₃(H₋₃L)₂]^{3-/0-}: Solutions of the compounds always contain a mixture of the D₃- and C₂-symmetric isomer. However, the crystal structures of C₂—K₃[Lu₃(H₋₃tacita)₂].20H₂O, C₂—K₃[Ho₃(H₋₃tacita)₂].17.5H₂O, D₃-[Hf₃(H₋₃tacitp)₂].9H₂O, D₃-K₃[Ho₃(H₋₃tacitp)₂].14.5H₂O, C₂—K₃[Lu₃(H₋₃macitp)₂].11 H₂O and C₂—K₃[Er₃(H₋₃macitp)₂].6.5H₂O exhibit only one diastereomer at a time in the crystal packing.

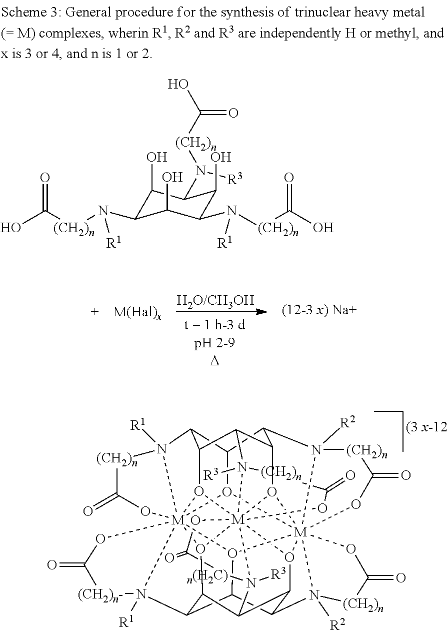

### DEFINITIONS

If chiral centres or other forms of isomeric centres are not otherwise defined in a compound according to the present invention, all forms of such stereoisomers, including enantiomers and diastereomers, are intended to be covered herein. Compounds containing chiral centres may be used as racemic mixture or as an enantiomerically enriched mixture or as a diastereomeric mixture or as a diastereomerically enriched mixture, or these isomeric mixtures may be separated using well-known techniques, and an individual stereoisomer maybe used alone.

### DESCRIPTION OF THE FIGURES

FIG. 1: Time course of contrast enhancement after intravenously administration of Na₃[Lu₃(H₋₃tacita)₂](Example 2).

FIG. 2: Region analysis of left heart chamber and respective signal-change time curve after administration of Na₃[Lu₃(H₋₃tacita)₂](Example 2).

FIG. 3: Crystal structure of C₂—[Lu₃(H₋₃tacita)₂]³⁻ (Example 2). The displacement ellipsoids are drawn at the 50% probability level; H(—N) hydrogen atoms are shown as spheres of arbitrary size; H(—C) hydrogen atoms are omitted for clarity. Only one position is shown for the disordered oxygen atom O43.

FIG. 4: Crystal structure of C₂—[Ho₃(H₋₃tacita)₂]³⁻ (Example 4). The displacement ellipsoids are drawn at the 50% probability level; H(—N) hydrogen atoms are shown as spheres of arbitrary size; H(—C) hydrogen atoms are omitted for clarity.

FIG. 5: Crystal structure of D₃-[Hf₃(H₋₃tacitp)₂](Example 13). The displacement ellipsoids are drawn at the 30% probability level; H(—N) hydrogen atoms are shown as spheres of arbitrary size; H(—C) hydrogen atoms are omitted for clarity. Only one position is shown for the disordered oxygen atom O65.

FIG. 6: Crystal structure of D₃-[Ho₃(H₋₃tacitp)₂]³⁻ (Example 15). The displacement ellipsoids are drawn at the 50% probability level; H(—N) hydrogen atoms are shown as spheres of arbitrary size; H(—C) hydrogen atoms are omitted for clarity. Only one position is shown for the disordered oxygen atom O26.

FIG. 7: Crystal structure of C₂—[Lu₃(H₋₃macitp)₂]³⁻ (Example 19). The displacement ellipsoids are drawn at the 30% probability level; H(—C) hydrogen atoms are omitted for clarity.

FIG. 8: Crystal structure of C₂—[Er₃(H₋₃macitp)₂]³⁻ (Example 22). The displacement ellipsoids are drawn at the 30% probability level; hydrogen atoms are omitted for clarity. Only one set of substituents is shown for the disordered groups bound to N2 and N4, respectively.

### EXPERIMENTAL PART

### Abbreviations

| | |
|---|---|
| br | broad signal (in NMR data) |
| d | doublet |
| ESI | electrospray ionisation |
| Hal | halogenide |
| HPLC | high performance liquid chromatography |
| ICP-OES | Inductively coupled plasma - optical emission spectrometry |
| ICP-MS | Inductively coupled plasma - mass spectrometry |
| L | ligand |
| MS | mass spectrometry |
| m | multiplet |
| M | metal |
| NMR | nuclear magnetic resonance spectroscopy |
| RT | room temperature |
| s | singlet |
| t | triplet |

### Materials and Instrumentation

The chemicals used for the synthetic work were of reagent grade quality and were used as obtained. Dowex 50 W-X2 (100-200 mesh, H⁺ form) and Dowex 1-X2 (50-100 mesh, Cl⁻ form) were from Sigma-Aldrich, the mixed bed ion exchange resin Amberlite MB-6113 from Merck. The starting materials 1,3,5-triamino-1,3,5-trideoxy-cis-inositol (taci)¹ and all-cis-2,4,6-tris(benzyloxy)-1,3,5-cyclohexanetriamine (tbca)² were prepared as described in the literature.

IR spectra were recorded on a Bruker Vector 22 FT IR spectrometer equipped with a Golden Gate ATR unit.

¹H and ¹³C{¹H}NMR spectra were measured in D₂O or CDCl₃, respectively (294 K, Bruker DRX Avance 400 MHz NMR spectrometer, resonance frequencies: 400.13 MHz for ¹H and 100.6 MHz for ¹³C). Chemical shifts are given in ppm relative to D₄-sodium (trimethylsilyl)propionate (D₂O) or tetramethylsilane (CDCl₃) as internal standards (δ=0 ppm). The pH* of the D₂O samples was adjusted using appropriate solutions of DCl and NaOD in D₂O. The term pH* refers to the direct pH-meter reading (Metrohm 713 pH meter) of the D₂O samples, using a Metrohm glass electrode with an aqueous (H₂O) Ag/AgCl-reference that was calibrated with aqueous (H₂O) buffer solutions.

Elemental analyses (C,H,N) were recorded on a LECO 900V or VARIO EL analyzer. Metal analyses were performed using ICP-OES methods.

For single crystal X-ray diffraction studies graphite monochromated Mo—K_{α} radiation (λ=0.71073 Å) was used throughout on a Bruker X8 Apex2 (T=100-153 K) or a Stoe IPDS (T=200 K) diffractometer. The structures were solved by direct methods (SHELXS-97) and refined by full-matrix, least squares calculations on F² (SHELXL-97).³ Anisotropic displacement parameters were refined for all non-hydrogen atoms except for the disordered O atoms in C₂—K₃[Ho₃(H₋₃tacita)₂].17.5H₂O and D₃-K₃[Ho₃ (H₋₃tacitp)₂].14.5H₂O (vide infra). Disorder: In the crystal structures of C₂—K₃[Lu₃ (H₋₃tacita)₂].20H₂O, C₂—K₃[Ho₃(H₋₃tacita)₂].17.5H₂O and C₂—K₃[Lu₃(H₋₃macitp)₂].11H₂O disorder of the solvent molecules and partially of the potassium counter ions was observed. Attempts to resolve the disorder were, however, not successful. The program SQUEEZE of the PLATON package⁴ was therefore applied and the electron density in the disordered regions was subtracted from the data sets. The final data sets contain the C₂—[Lu₃(H₋₃tacita)₂]³⁻ and the C₂—[Ho₃(H₋₃tacita)₂]³⁻ anions and the C₂—K₃[Lu₃(H₋₃macitp)₂].3H₂O entity, respectively. The elemental formulae of the crystal structures were deduced from the amount of electrons that was subtracted in each case. The oxygen atoms O43 in C₂—K₃[Lu₃(H₋₃tacita)₂].20H₂O as well as O26 in D₃-K₃[Ho₃(H₋₃tacitp)₂].14.5H₂O were found to be distributed over two sites (A and B) with occupancies of 50%. A similar disorder was found for O65 in D₃-[Hf₃(H₋₃tacitp)₂].9H₂O with occupancies of 72% and 28% for the two sites A and B. In D₃-K₃[Ho₃(H₋₃tacitp)₂].14.5H₂O the potassium counter ion K3 was distributed over three sites with occupancies of 50% (A), 35% (C) and 15% (B), respectively. The complex anions in C₂—K₃[Lu₃(H₋₃macitp)₂].11 H₂O and C₂—K₃[Er₃ (H₋₃macitp)₂].6.5H₂O were located on a crystallographic mirror plane resulting in either case in a 1:1 disorder of two propionate pendant arms and two methyl groups, respectively. Treatment of hydrogen atoms: Calculated positions (riding model) were generally used for H(—C) atoms. The H(—N) positions of C₂—K₃[Lu₃(H₋₃tacita)₂].20H₂O and C₂—K₃[Ho₃(H₋₃tacita)₂].17.5H₂O were also calculated. All other H(—N) and H(—O) positions were refined using isotropic displacement parameters with Uᵢₛₒ of the H atoms being set to 1.2 or 1.5×U_{eq} of the pivotal N or O atom, respectively. Furthermore, restraints were used for the N—H and O—H distances. Not all of the H(—O) atoms of the solvent molecules in the crystal structures containing crystal water could be located and the corresponding positions were therefore not considered in the refinement.

Mass spectra were measured on a Waters LC/MS spectrometer equipped with a ZQ 4000-ESI mass spectrometer (single quadrupol).

### Intermediates

### Intermediate 1

### 1,3,5-Triamino-1,3,5-trideoxy-cis-inositol-tri-N,N′,N″-acetic acid (H₃tacita)

all-cis-2,4,6-Tris(benzyloxy)-1,3,5-cyclohexanetriamine (3.0 g, 6.7 mmol) was dissolved in dichloromethane (120 mL) and N,N-diisopropylethylamine (3.3 mL, 20.1 mmol) was added. tert-Butyl bromoacetate (3.4 mL, 23.5 mmol) was added dropwise to the solution which was stirred for three days at ambient temperature afterwards. The solvent was completely removed and the residue was dissolved in methanol (50 mL). After addition of 6 M hydrochloric acid (300 mL) the suspension was heated to reflux for 24 h. The resulting solution was extracted twice with dichloromethane and the aqueous layer was evaporated to dryness. The white solid was dissolved in water (50 mL) and the pH was adjusted to 5.5 using sodium hydroxide (40%) to get a white precipitate that was filtered off, washed with ethanol, and dried in vacuo.

Yield: 2.5 g (92%) H₃tacita.3H₂O.

¹H NMR (D₂O, pH* <1) δ 3.85 (t, J=3 Hz, 3H), 4.24 (s, 6H), 4.78 (t, J=3 Hz, 3H).

¹³C NMR (D₂O, pH* <1) δ 45.7, 57.6, 64.4, 169.3.

¹H NMR (D₂O, pH* >13) δ 2.57 (m, 3H), 3.32 (s, 6H), 4.12 (m, 3H).

¹³C NMR (D₂O, pH* >13) δ 51.9, 60.5, 71.3, 182.6.

Anal. Calcd (%) for C₁₂H₂₁N₃O₉.3H₂O (405.36): C, 35.56; H, 6.71; N, 10.37. Found: C, 35.36; H, 6.49; N, 10.25.

IR (cm⁻¹): 607, 632, 679, 793, 914, 936, 978, 1012, 1133, 1214, 1283, 1328, 1371, 1404, 1574, 2744, 3054, 3421.

### Intermediate 2

### 1,3,5-Trideoxy-1,3,5-tris(methylamino)-cis-inositol-tri-N,N′,N″-acetic acid (H₃macita)

H₃tacita.3H₂O (1.8 g, 4.4 mmol) was suspended in water (200 mL) and the pH was adjusted to ˜1 using concentrated hydrochloric acid. To the resulting solution was added a formaldehyde solution (37%, 70 mL, 936 mmol) and platinum(IV) oxide (600 mg) as catalyst. The reaction mixture was hydrogenated in an autoclave at 5 atm H₂. After 15 days, the catalyst was filtered off and the filtrate was concentrated to dryness. The residue was dissolved twice in a 1:1 mixture of water and formic acid (30 mL) and evaporated to dryness again. The remaining solid was taken up in few hydrochloric acid (0.5 M) and sorbed on DOWEX 50. The column was washed successively with water (1 L), 0.5 M hydrochloric acid (1 L), and 3 M hydrochloric acid (2 L). The 3 M fraction containing the product was evaporated to dryness and the light yellow solid was dried in vacuo.

Yield: 2.1 g (91%) H₃macita.3HCl.H₂O.

¹H NMR (D₂O, pH* <2) δ 3.30 (s, 9H), 4.12 (m, 3H), 4.38 (s, 6H), 4.91 (m, 3H).

¹³C NMR (D₂O, pH* <2) δ 43.6, 56.7, 65.1, 65.3, 170.9.

Anal. Calcd (%) for C₁₅H₂₇N₃O₉.3HCl.H₂O (520.79): C, 34.59; H, 6.19; N, 8.07. Found: C, 34.71; H, 6.23; N, 8.13.

IR (cm⁻¹): 603, 662, 686, 836, 1006, 1099, 1205, 1410, 1725, 2961.

### Intermediate 3-1

### 1,3,5-Triamino-1,3,5-trideoxy-cis-inositol-tri-N,N′,N″-propionitrile (tacitpn)

taci (2.0 g, 11.3 mmol) was dissolved in methanol (100 mL) and acrylonitrile (7.4 mL, 0.11 mol) was added. The solution was stirred for 24 h at ambient temperature. The solvent was removed, the residue washed successively with diethyl ether and hexane and the white solid was dried in vacuo.

Yield: 3.9 g (97%) tacitpn.0.2H₂O.0.5MeOH. Single crystals suitable for X-ray analysis were obtained by evaporation of a concentrated solution of tacitpn in methanol.

¹H NMR (D₂O) δ 2.72 (m, 9H), 3.03 (t, J=7 Hz, 6H), 4.23 (t, J=3 Hz, 3H).

¹³C NMR (D₂O) δ 20.5, 43.4, 60.1, 72.0, 123.2.

Anal. Calcd (%) for C₁₅H₂₄N₆O₃.0.2H₂O.0.5MeOH (356.01): C, 52.29; H, 7.47; N, 23.61. Found: C, 52.23; H, 7.23; N, 23.40.

IR (cm⁻¹): 602, 754, 843, 902, 1072, 1113, 1252, 1352, 1425, 1987, 2067, 2248, 2924, 3103, 3268.

MS (ES⁺): m/z (%) 337.5 (100) {tacitpn+H}⁺.

MS (ES⁻): m/z (%) 335.6 (100) {tacitpn−H}⁻.

### Intermediate 3-2

### 1,3,5-Triamino-1,3,5-trideoxy-cis-inositol-tri-N,N′,N″-propionic acid (H₃tacitp)

tacitpn (3.8 g, 10.7 mmol) was dissolved in sodium hydroxide (10.3 g of a 25% solution, 64.4 mmol) and heated to reflux for 4 h. The solvent was removed and the residue was taken up in 1 M hydrochloric acid (5 mL) and sorbed on DOWEX 50. The column was washed with water (1 L), 0.25 M hydrochloric acid (1 L), 1 M hydrochloric acid (1 L) and the product was eluted with 3 M hydrochloric acid (1 L). The solvent was removed and the solid dried in vacuo.

Yield: 5.1 g (86%) H₃tacitp.3HCl.3H₂O.

¹H NMR (D₂O) δ 2.43 (t, J=7 Hz, 6H), 2.61 (m, 3H), 2.89 (t, J=7 Hz, 6H), 4.26 (m, 3H).

¹³C NMR (D₂O) δ 40.3, 44.7, 60.5, 71.8, 184.2.

Anal. Calcd (%) for C₁₅H₂₇N₃O₉.3HCl.3H₂O (556.82): C, 32.36; H, 6.52; N, 7.55. Found: C, 32.56; H, 6.31; N, 7.64.

IR (cm⁻¹): 1073, 1111, 1308, 1409, 1458, 1571, 2903.

MS (ES⁺): m/z (%) 441.4 (100) {H₂tacitp+2Na}⁺, 394.2 (75) {H₃tacitp+H}⁺.

MS (ES⁻): m/z (%) 392.3 (100) {H₃tacitp−H}⁻.

### Intermediate 4

### 1,3,5-Trideoxy-1,3,5-tris(methylamino)-cis-inositol-tri-N,N′,N″-propionic acid (H₃macitp)

H₃tacitp.3HCl.3H₂O (400 mg, 0.7 mmol) was dissolved in a formaldehyde solution (37%, 25 mL, 334 mmol) and a small amount of Pd (10%)/C was added. The reaction mixture was hydrogenated in an autoclave at 50 atm H₂ for 4 days at RT. The reaction mixture was filtered off and the filtrate concentrated to dryness. The residue was dissolved twice in a 1:1 mixture of water and formic acid (30 mL) and evaporated to dryness again. The remaining solid was taken up in 3 M hydrochloric acid (10 mL) and sorbed on DOWEX 50. The column was washed successively with 0.5 M hydrochloric acid (1 L), 1 M hydrochloric acid (1 L) and 3 M hydrochloric acid (1 L). The 3 M fraction containing the product was evaporated to dryness and the solid was dried in vacuo.

Yield: 320 mg (71%) H₃macitp.3HCl.4.5H₂O.

¹H NMR (D₂O) δ 3.04 (t, J=7 Hz, 6H), 3.15 (s, 9H), 3.67 (m, 3H), 3.78 (t, J=7 Hz, 6H), 5.04 (m, 3H).

¹³C NMR (D₂O) δ 3.6, 34.3, 45.5, 57.9, 58.6, 169.9.

Anal. Calcd (%) for C₁₈H₃₃N₃O₉.3HCl.4.5H₂O (625.92): C, 34.54; H, 7.25; N, 6.71. Found: C, 34.20; H, 6.86; N, 6.71.

IR (cm⁻¹): 647, 798, 988, 1099, 1138, 1188, 1401, 1714, 1943, 2008, 2115, 2165, 2189, 2927.

### EXAMPLES

### Example 1

### Hf₃(H₋₃tacita)₂

Hafnium(IV) chloride (594 mg, 1.9 mmol) was dissolved in water (20 mL). H₃tacita.3H₂O (0.5 g, 1.2 mmol) was added and the pH was adjusted to ˜2.5 (1 M sodium hydroxide). The solution was heated to reflux for 20 h. The reaction mixture was filtered and the filtrate was sorbed on DOWEX 50 (H⁺-form). The product was eluted with water, the solvent removed and the white solid dried in vacuo.

Yield: 65 mg (8%) [Hf₃(H₋₃tacita)₂].6.5H₂O as a 2:1 mixture (deduced from ¹H NMR) of the C₂- and D₃-symmetric complex species.

¹H NMR (D₂O, pH* <2) δ 3.72-3.78 ([3×C₂+D₃]-CHₐₓ, 6H), 3.90-3.93 ([3×C₂+D₃]-CH₂^{a}, 6H), 4.12-4.21 ([3×C₂+D₃]-CH₂^{b}, 6H), 4.87 (m, [C₂]—CH_{eq}, 1.3H), 4.97 ([C₂+D₃]-CH_{eq}, 3.3H), 5.08 (m, [C₂]—CH_{eq}, 1.3H), 6.11-6.18 ([3×C₂+D₃]-NH, 6H).

¹³C NMR (D₂O, pH* <2) δ 51.7, 51.8, 51.9, 52.0, 62.56, 62.60, 62.9 (×2), 74.3, 76.68, 76.69, 79.0, 185.0, 185.1, 185.2, 185.3.

Anal. Calcd (%) for C₂₄H₃₀Hf₃N₆O₁₈.6.5H₂O (1343.09): C, 21.46; H, 3.23; N, 6.26; Hf, 39.87. Found: C, 22.06; H, 3.25; N, 6.07; Hf, 39.47.

IR (cm⁻¹): 513, 522, 549, 559, 570, 580, 652, 716, 819, 916, 960, 1016, 1087, 1114, 1303, 1348, 1504, 1634, 2961, 3159.

MS (ES⁺): m/z (%) 1249.2 (100) {[Hf₃(H₋₃tacita)₂]+Na}⁺, 1227.2 (14) {[Hf₃(H₋₃tacita)₂]+H}⁺.

MS (ES⁻): m/z (%) 1225.3 (100) {[Hf₃(H₋₃tacita)₂]−H}⁻.

### Example 2

### Na₃[Lu₃(H₋₃tacita)₂]

H₃tacita.3H₂O (1.0 g, 2.5 mmol) was suspended in methanol (120 mL). Sodium hydroxide (12.5 mL of a 1 M solution in methanol, 12.5 mmol) was added to get a clear solution to which were dropped 1.5 eq of lutetium(III) chloride hexahydrate (1.5 g, 3.9 mmol) dissolved in methanol (20 mL). The suspension was heated to reflux for 2 h and reduced to a volume of 50 mL. The white solid was filtered off after cooling and dissolved in water (30 mL) at pH ˜9 (adjusted with 1 M sodium hydroxide). The solution was heated to reflux again for 1 h, filtered and the product was precipitated from the filtrate after cooling with ethanol (150 mL). The white solid was filtered off and dried in vacuo.

Yield: 1.3 g (76%) Na₃[Lu₃(H₋₃tacita)₂].5.5H₂O as a 3:2 mixture (deduced from ¹H NMR) of the C₂- and D₃-symmetric complex species. Single crystals of the composition C₂—K₃[Lu₃(H₋₃tacita)₂].20H₂O were obtained by slow evaporation of an aqueous solution of the complex (pH ˜11, potassium hydroxide used in the synthesis).

¹H NMR (D₂O, pH* ˜7) δ 2.90 (m, [C₂]—CHₐₓ, 1.2H), 2.91 (m, [C₂]—CHₐₓ, 1.2H), 2.95 (m, [D₃]-CHₐₓ, 2.4H), 2.97 (m, [C₂]—CHₐₓ, 1.2H), 3.34 (br, [D₃+3×C₂]—NH, 6H), 3.43-3.53 ([D₃+3×C₂]—CH₂^{a}, 6H), 3.70-3.80 ([D₃+3×C₂]—CH₂^{b}, 6H), 4.10 (m, [C₂]—CH_{eq}, 1.2H), 4.25 (m, [C₂+D₃]-CH_{eq}, 3.6H), 4.40 (m, [C₂]—CH_{eq}, 1.2H).

¹³C NMR (D₂O, pH* ˜7) δ 50.3, 50.4 (D₃), 50.6, 50.7, 63.57 (D₃), 63.62, 63.8, 63.9, 70.2, 73.0, 73.1 (D₃), 75.9, 186.89, 186.95 (D₃), 186.97, 187.03.

Anal. Calcd (%) for C₂₄H₃₀Lu₃N₆Na₃O₁₈.5.5H₂O (1383.48): C, 20.84; H, 2.99; N, 6.08; Lu, 37.94; Na, 4.99. Found: C, 20.95; H, 3.18; N, 6.05; Lu, 38.07; Na, 5.02.

IR (cm⁻¹): 513, 527, 540, 566, 580, 594, 613, 635, 710, 793, 863, 888, 946, 995, 1059, 1114, 1141, 1259, 1320, 1376, 1434, 1582, 2848, 3268.

MS (ES⁺): m/z (%) 1307.8 (100) {[Lu₃(H₋₃tacita)₂]+4Na}⁺.

### Crystal Data and Structure Refinement:

| Empirical formula | C₂₄H₇₀K₃Lu₃N₆O₃₈ | |
|---|---|---|
| Formula weight | 1693.07 | |
| Temperature | 123(2) K | |
| Wavelength | 0.71073 Å | |
| Crystal system | Triclinic | |
| Space group | P-1 | |
| Unit cell dimensions | a = 12.3837(7) Å | α = 76.977(2)°. |
| | b = 13.9778(8) Å | β = 69.410(2)°. |
| | c = 15.8816(9) Å | γ = 89.694(3)°. |

| | |
|---|---|
| Volume | 2499.0(2) Å³ |
| Z | 2 |
| Density (calculated) | 2.250 Mg/m³ |
| Absorption coefficient | 6.244 mm⁻¹ |
| F(000) | 1660 |
| Crystal size | 0.56 × 0.20 × 0.13 mm³ |
| Theta range for data collection | 1.41 to 35.00°. |
| Index ranges | −19 <= h <= 19, −22 <= |
| | k <= 22, −25 <= l <= 21 |
| Reflections collected | 102114 |
| Independent reflections | 21974 [R(int) = 0.0273] |
| Completeness to theta = 35.00° | 99.9% |
| Absorption correction | Semi-empirical from equivalents |
| Max. and min. transmission | 0.4974 and 0.1277 |
| Refinement method | Full-matrix least-squares on F² |
| Data/restraints/parameters | 21974/0/466 |
| Goodness-of-fit on F² | 1.058 |
| Final R indices [I > 2sigma(I)] | R₁ = 0.0159, wR₂ = 0.0397 |
| R indices (all data) | R₁ = 0.0179, wR₂ = 0.0404 |
| Largest diff. peak and hole | 1.631 and −1.227 e · Å⁻³ |

Atomic coordinates (×10⁴) and equivalent isotropic displacement parameters (Å²×10³). U(eq) is defined as one third of the trace of the orthogonalized U^{ij} tensor.

| | | | |
|---|---|---|---|
| x | y | z | U (eq) |

| | | | | | | |
|---|---|---|---|---|---|---|
| Lu (1) | 2176 | (1) | 1754 | (1) | 7638 (1) | 9 (1) |
| Lu (2) | 1675 | (1) | 2108 | (1) | 5508 (1) | 9 (1) |
| Lu (3) | 69 | (1) | 176 | (1) | 7441 (1) | 9 (1) |
| C (11) | −898 | (1) | 2243 | (1) | 6873 (1) | 11 (1) |
| O (11) | −114 | (1) | 1581 | (1) | 6514 (1) | 11 (1) |
| C (12) | −1434 | (1) | 1900 | (1) | 7935 (1) | 11 (1) |
| N (12) | −1807 | (1) | 836 | (1) | 8180 (1) | 12 (1) |
| C (121) | −2696 | (1) | 577 | (1) | 7833 (1) | 14 (1) |
| C (122) | −2209 | (1) | 92 | (1) | 7017 (1) | 14 (1) |
| O (123) | −2731 | (1) | 163 | (1) | 6464 (1) | 25 (1) |
| O (124) | −1323 | (1) | −392 | (1) | 6972 (1) | 16 (1) |
| C (13) | −554 | (1) | 1992 | (1) | 8398 (1) | 11 (1) |
| O (13) | 264 | (1) | 1281 | (1) | 8234 (1) | 11 (1) |
| C (14) | 73 | (1) | 3028 | (1) | 8075 (1) | 11 (1) |
| N (14) | 1021 | (1) | 2935 | (1) | 8450 (1) | 12 (1) |
| C (141) | 1700 | (1) | 3846 | (1) | 8327 (1) | 16 (1) |
| C (142) | 3000 | (1) | 3757 | (1) | 7925 (1) | 18 (1) |
| O (143) | 3658 | (1) | 4473 | (1) | 7843 (1) | 31 (1) |
| O (144) | 3360 | (1) | 2969 | (1) | 7692 (1) | 19 (1) |
| C (15) | 603 | (1) | 3393 | (1) | 7010 (1) | 11 (1) |
| O (15) | 1601 | (1) | 2905 | (1) | 6636 (1) | 11 (1) |
| C (16) | −295 | (1) | 3284 | (1) | 6563 (1) | 12 (1) |
| N (16) | 366 | (1) | 3488 | (1) | 5547 (1) | 12 (1) |
| C (161) | −334 | (1) | 3461 | (1) | 4970 (1) | 14 (1) |
| C (162) | −191 | (1) | 2548 | (1) | 4571 (1) | 14 (1) |
| O (163) | −978 | (1) | 2317 | (1) | 4308 (1) | 22 (1) |
| O (164) | 713 | (1) | 2096 | (1) | 4499 (1) | 15 (1) |
| C (21) | 2282 | (1) | −147 | (1) | 5744 (1) | 10 (1) |
| O (21) | 1347 | (1) | 453 | (1) | 5960 (1) | 10 (1) |
| C (22) | 3428 | (1) | 498 | (1) | 5235 (1) | 11 (1) |
| N (22) | 3203 | (1) | 1268 | (1) | 4515 (1) | 11 (1) |
| C (221) | 4188 | (1) | 1948 | (1) | 3857 (1) | 16 (1) |
| C (222) | 3889 | (1) | 3020 | (1) | 3719 (1) | 17 (1) |
| O (223) | 4581 | (1) | 3644 | (1) | 3062 (1) | 27 (1) |
| O (224) | 2953 | (1) | 3232 | (1) | 4296 (1) | 17 (1) |
| C (23) | 3802 | (1) | 991 | (1) | 5876 (1) | 11 (1) |
| O (23) | 3107 | (1) | 1766 | (1) | 6106 (1) | 11 (1) |
| C (24) | 3786 | (1) | 238 | (1) | 6754 (1) | 11 (1) |
| N (24) | 3961 | (1) | 830 | (1) | 7372 (1) | 12 (1) |
| C (241) | 4044 | (1) | 262 | (1) | 8246 (1) | 18 (1) |
| C (242) | 2993 | (2) | 319 | (1) | 9089 (1) | 31 (1) |
| O (43A) | 3150 | (3) | −147 | (3) | 9851 (2) | 41 (1) |
| O (43B) | 2586 | (3) | −427 | (3) | 9770 (2) | 41 (1) |
| O (244) | 2336 | (1) | 1002 | (1) | 9030 (1) | 17 (1) |
| C (25) | 2632 | (1) | −393 | (1) | 7269 (1) | 11 (1) |
| O (25) | 1765 | (1) | 186 | (1) | 7671 (1) | 11 (1) |
| C (26) | 2281 | (1) | −889 | (1) | 6624 (1) | 11 (1) |
| N (26) | 1062 | (1) | −1320 | (1) | 7109 (1) | 12 (1) |
| C (261) | 856 | (1) | −2088 | (1) | 7963 (1) | 15 (1) |
| C (262) | 204 | (1) | −1737 | (1) | 8837 (1) | 19 (1) |
| O (263) | 329 | (2) | −2160 | (1) | 9574 (1) | 41 (1) |
| O (264) | −450 | (1) | −1040 | (1) | 8772 (1) | 16 (1) |

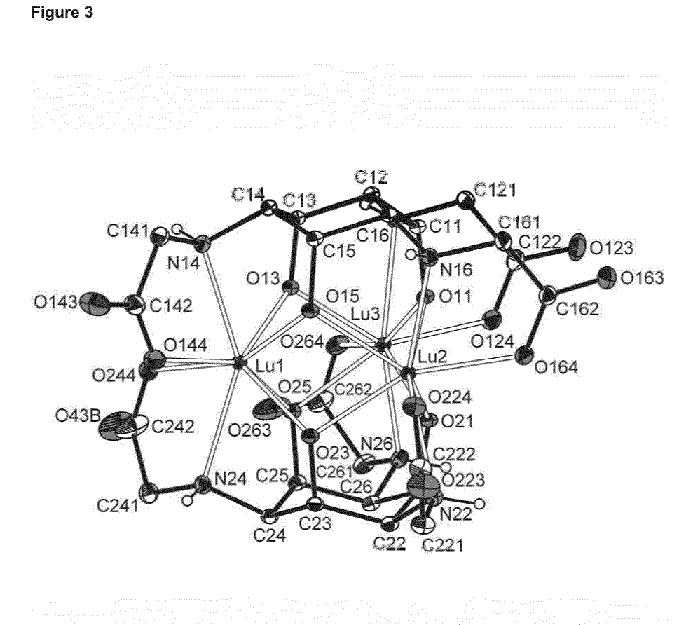

FIG. 3 shows the crystal structure.

### Example 3

### Na₃[Gd₃(H₋₃tacita)₂]

The complex was prepared from H₃tacita.3H₂O (220 mg, 0.5 mmol) and gadolinium(III) chloride hexahydrate (280 mg, 0.8 mmol) by following the protocol for the preparation of the lutetium complex Na₃[Lu₃(H₋₃tacita)₂].

Yield: 237 mg (64%) as Na₃[Gd₃(H₋₃tacita)₂].8H₂O.

Anal. Calcd (%) for C₂₄H₃₀Gd₃N₆Na₃O₁₈.8H₂O (1375.37): C, 20.96; H, 3.37; N, 6.11; Gd, 34.30; Na, 5.02. Found: C, 20.99; H, 3.55; N, 6.13; Gd, 34.44; Na, 5.04.

IR (cm⁻¹): 515, 522, 544, 561, 570, 586, 614, 646, 704, 783, 867, 876, 940, 995, 1058, 1113, 1139, 1263, 1320, 1382, 1428, 1574, 2826, 3232.

MS (ES⁺): m/z (%) 1255.0 (100) {[Gd₃(H₋₃tacita)₂]+4Na}⁺, 1274.9 (8) {[Gd₃ (H₋₃tacita)₂]+5Na−H}⁺.

MS (ES⁻): m/z (%) 1208.9 (100) {[Gd₃(H₋₃tacita)₂]+2Na}⁻, 1186.1 (25) {[Gd₃ (H₋₃tacita)₂]+Na+H}⁻, 1230.9 (20) {[Gd₃(H₋₃tacita)₂]+3Na−H}⁻.

### Example 4

### Na₃[Ho₃(H₋₃tacita)₂]

The complex was prepared according to the protocol for the lutetium complex Na₃[Lu₃ (H₋₃tacita)₂] using H₃tacita.3H₂O (150 mg, 0.4 mmol) and holmium(III) chloride (146 mg, 0.5 mmol) as starting material.

Yield: 86 mg (33%) Na₃[Ho₃(H₋₃tacita)₂].8H₂O. Single crystals of the composition C₂—K₃[Ho₃(H₋₃tacita)₂].17.5H₂O were obtained by slow evaporation of an aqueous solution of the complex (pH ˜11, potassium hydroxide used in the synthesis).

Anal. Calcd (%) for C₂₄H₃₀Ho₃N₆Na₃O₁₈.8H₂O (1398.41): C, 20.61; H, 3.32; N, 6.01. Found: C, 20.43; H, 2.87; N, 5.53.

MS (ES⁺): m/z (%) 1276.8 (100) {[Ho₃(H₋₃tacita)₂]+4Na}⁺, 1254.9 (13) {[Ho₃ (H₋₃tacita)₂]+3Na+H}⁺, 1232.9 (5) {[Ho₃(H₋₃tacita)₂]+2Na+2H}⁺.

MS (ES⁻): m/z (%) 593.1 (100) {[Ho₃(H₋₃tacita)₂]+H}²⁻, 604.1 (20) {[Ho₃(H₋₃tacita)₂]+Na}²⁻, 1187.1 (5) {[Ho₃(H₋₃tacita)₂]+2H}⁻, 1209.1 (2) {[Ho₃(H₋₃tacita)₂]+H+Na}⁻.

### Crystal Data and Structure Refinement:

| Empirical formula | C₂₄H₆₅Ho₃K₃N₆O_{35.50} | |
|---|---|---|
| Formula weight | 1617.91 | |
| Temperature | 153(2) K | |
| Wavelength | 0.71073 Å | |
| Crystal system | Triclinic | |
| Space group | P-1 | |
| Unit cell dimensions | a = 12.4835(4) Å | α = 103.2985(16)°. |
| | b = 13.9625(4) Å | β = 110.4896(14)°. |
| | c = 15.8312(5) Å | γ = 90.5804(17)°. |

| | |
|---|---|
| Volume | 2503.04(13) Å³ |
| Z | 2 |
| Density (calculated) | 2.147 Mg/m³ |
| Absorption coefficient | 5.053 mm⁻¹ |
| F(000) | 1586 |
| Crystal size | 0.38 × 0.28 × 0.24 mm³ |
| Theta range for data collection | 1.42 to 37.50°. |
| Index ranges | −21 <= h <= 21, −23 <= |
| | k <= 23, −27 <= l <= 27 |
| Reflections collected | 78329 |
| Independent reflections | 26211 [R(int) = 0.0280] |
| Completeness to theta = 37.50° | 99.5% |
| Absorption correction | Semi-empirical from equivalents |
| Max. and min. transmission | 0.3768 and 0.2497 |
| Refinement method | Full-matrix least-squares on F² |
| Data/restraints/parameters | 26211/0/459 |
| Goodness-of-fit on F² | 1.088 |
| Final R indices [I > 2sigma(I)] | R₁ = 0.0349, wR₂ = 0.0909 |
| R indices (all data) | R₁ = 0.0397, wR₂ = 0.0932 |
| Largest diff. peak and hole | 9.151 and −2.250 e · A⁻³ |

Atomic coordinates (×10⁴) and equivalent isotropic displacement parameters (Å²×10³). U(eq) is defined as one third of the trace of the orthogonalized U^{ij} tensor.

| | x | y | z | U (eq) |
|---|---|---|---|---|
| Ho (1) | 2804 (1) | 3233 (1) | 7636 (1) | 11 (1) |
| Ho (2) | 3319 (1) | 2881 (1) | 5469 (1) | 10 (1) |
| Ho (3) | 4959 (1) | 4865 (1) | 7456 (1) | 10 (1) |
| C (11) | 1205 (2) | 4005 (2) | 5857 (2) | 12 (1) |
| O (11) | 1876 (2) | 3226 (2) | 6079 (1) | 13 (1) |
| C (12) | 1586 (2) | 4509 (2) | 5225 (2) | 12 (1) |
| N (12) | 1786 (2) | 3745 (2) | 4486 (2) | 13 (1) |
| C (121) | 793 (2) | 3065 (2) | 3821 (2) | 17 (1) |
| C (122) | 1068 (2) | 1989 (2) | 3648 (2) | 17 (1) |
| O (123) | 366 (2) | 1374 (2) | 2978 (2) | 29 (1) |
| O (124) | 1987 (2) | 1764 (2) | 4206 (2) | 19 (1) |
| C (13) | 2731 (2) | 5164 (2) | 5743 (2) | 12 (1) |
| O (13) | 3656 (2) | 4574 (1) | 5949 (1) | 12 (1) |
| C (14) | 2734 (2) | 5905 (2) | 6627 (2) | 12 (1) |
| N (14) | 3936 (2) | 6364 (2) | 7118 (2) | 14 (1) |
| C (141) | 4137 (3) | 7140 (2) | 7980 (2) | 17 (1) |
| C (142) | 4761 (3) | 6792 (2) | 8852 (2) | 19 (1) |
| O (143) | 4590 (3) | 7188 (3) | 9582 (2) | 38 (1) |
| O (144) | 5445 (2) | 6132 (2) | 8807 (2) | 19 (1) |
| C (15) | 2382 (2) | 5401 (2) | 7274 (2) | 13 (1) |
| O (15) | 3240 (2) | 4835 (2) | 7675 (1) | 13 (1) |
| C (16) | 1228 (2) | 4764 (2) | 6741 (2) | 13 (1) |
| N (16) | 1017 (2) | 4169 (2) | 7349 (2) | 14 (1) |
| C (161) | 920 (2) | 4747 (3) | 8217 (2) | 20 (1) |
| C (162) | 1959 (3) | 4727 (3) | 9070 (2) | 27 (1) |
| O (163) | 2144 (4) | 5446 (4) | 9771 (3) | 59 (1) |
| O (164) | 2617 (2) | 4054 (2) | 9041 (2) | 19 (1) |
| C (21) | 5886 (2) | 2768 (2) | 6860 (2) | 12 (1) |
| O (21) | 5124 (2) | 3428 (1) | 6503 (1) | 12 (1) |
| C (22) | 5277 (2) | 1717 (2) | 6542 (2) | 13 (1) |
| N (22) | 4632 (2) | 1493 (2) | 5523 (2) | 14 (1) |
| C (221) | 5337 (2) | 1512 (2) | 4947 (2) | 16 (1) |
| C (222) | 5231 (2) | 2432 (2) | 4569 (2) | 16 (1) |
| O (223) | 6041 (2) | 2669 (2) | 4331 (2) | 25 (1) |
| O (224) | 4333 (2) | 2875 (2) | 4468 (2) | 18 (1) |
| C (23) | 4384 (2) | 1602 (2) | 6989 (2) | 13 (1) |
| O (23) | 3392 (2) | 2074 (1) | 6611 (1) | 13 (1) |
| C (24) | 4913 (2) | 1964 (2) | 8056 (2) | 13 (1) |
| N (24) | 3982 (2) | 2039 (2) | 8438 (2) | 14 (1) |
| C (241) | 3308 (2) | 1118 (2) | 8309 (2) | 18 (1) |
| C (242) | 2021 (3) | 1211 (2) | 7979 (2) | 20 (1) |
| O (243) | 1376 (2) | 500 (2) | 7942 (2) | 34 (1) |
| O (244) | 1651 (2) | 1990 (2) | 7740 (2) | 25 (1) |
| C (25) | 5548 (2) | 3008 (2) | 8391 (2) | 13 (1) |
| O (25) | 4744 (2) | 3729 (1) | 8245 (1) | 12 (1) |
| C (26) | 6417 (2) | 3105 (2) | 7924 (2) | 13 (1) |
| N (26) | 6824 (2) | 4175 (2) | 8197 (2) | 13 (1) |
| C (261) | 7722 (2) | 4429 (2) | 7860 (2) | 15 (1) |
| C (262) | 7269 (3) | 4927 (2) | 7052 (2) | 17 (1) |
| O (263) | 7810 (3) | 4843 (2) | 6508 (2) | 31 (1) |
| O (264) | 6406 (2) | 5419 (2) | 6999 (2) | 19 (1) |

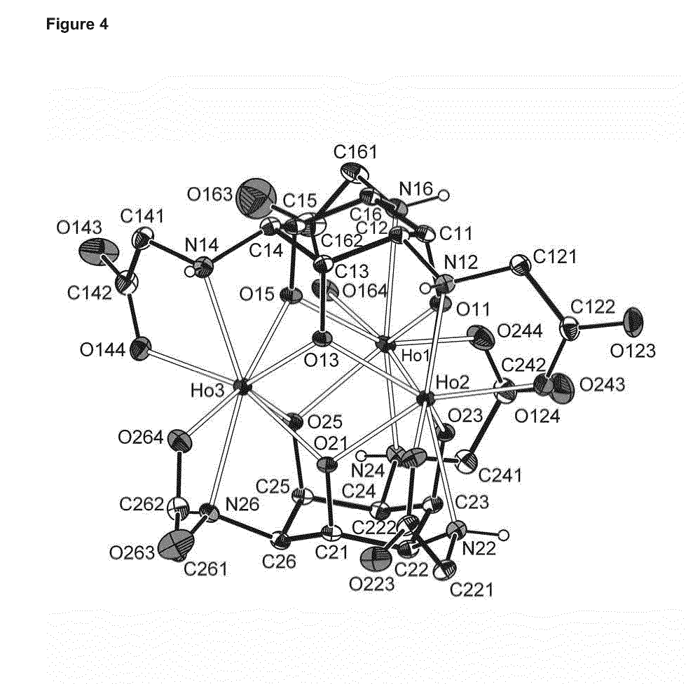

FIG. 4 shows the crystal structure.

### Example 5

### Na₃[Er₃(H₋₃tacita)₂]

The complex was prepared according to the protocol for the lutetium complex Na₃[Lu₃ (H₋₃tacita)₂] using H₃tacita.3H₂O (150 mg, 0.4 mmol) and erbium(III) chloride hexahydrate (215 mg, 0.6 mmol) as starting material.

Yield: 155 mg (57%) as Na₃[Er₃(H₋₃tacita)₂].12H₂O.

Anal. Calcd (%) for C₂₄H₃₀Er₃N₆Na₃O₁₈.12H₂O (1477.45): C, 19.51; H, 3.68; N, 5.69. Found: C, 19.46; H, 3.21; N, 5.26.

IR (cm⁻¹): 510, 526, 540, 552, 570, 590, 629, 686, 703, 793, 875, 885, 943, 999, 1063, 1112, 1139, 1259, 1320, 1383, 1435, 1566, 2866, 3252.

MS (ES⁺): m/z (%) 653.3 (100) {[Er₃(H₋₃tacita)₂]+5Na}²⁺, 1283.8 (8) {[Er₃ (H₋₃tacita)₂]+4Na}⁺, 1261.8 (1) {[Er₃(H₋₃tacita)₂]+3Na+H}⁺.

MS (ES⁻): m/z (%) 1193.8 (100) {[Er₃(H₋₃tacita)₂]+2H}⁻, 1215.8 (32) {[Er₃ (H₋₃tacita)₂]+Na+H}⁻.

### Example 6

### Na₃[Yb₃(H₋₃tacita)₂]

The complex was prepared from H₃tacita.3H₂O (1.3 g, 3.2 mmol) and ytterbium(III) chloride hexahydrate (1.9 g, 4.9 mmol) by following the protocol for the preparation of the lutetium complex Na₃[Lu₃(H₋₃tacita)₂].

Yield: 1.7 g (74%) as Na₃[Yb₃(H₋₃tacita)₂].9H₂O.

Anal. Calcd (%) for C₂₄H₃₀N₆Na₃O₁₈Yb₃.9H₂O (1440.79): C, 20.01; H, 3.36; N, 5.83; Yb, 36.03; Na, 4.79. Found: C, 20.47; H, 3.65; N, 6.08; Yb, 35.73; Na, 5.02.

IR (cm⁻¹): 508, 526, 547, 585, 611, 632, 674, 698, 791, 875, 890, 944, 996, 1060, 1111, 1139, 1262, 1322, 1378, 1432, 1583, 2848, 3269.

MS (ES⁺): m/z (%) 1301.9 (100) {[Yb₃(H₋₃tacita)₂]+4Na}⁺, 1278.8 (13) {[Yb₃(H₋₃tacita)₂]+3Na+H}⁺.

MS (ES⁻): m/z (%) 1254.9 (100) {[Yb₃(H₋₃tacita)₂]+2Na}⁻, 1233.1 (45) {[Yb₃(H₋₃tacita)₂]+Na+H}⁻.

### Example 7

### Hf₃(H₋₃macita)₂

Hafnium(IV) chloride (205 mg, 0.6 mmol) was dissolved in water (35 mL). H₃macita.3HCl.H₂O (250 mg, 0.5 mmol) was added and the pH was adjusted to ˜3 (1 M sodium hydroxide). The solution was heated to reflux for 24 h and allowed to stand at RT in an open beaker for one day afterwards. The solid was filtered off and dried in vacuo.

Yield: 50 mg (14%) [Hf₃(H₋₃macita)₂].12H₂O (C₂-symmetric complex as major species).

¹H NMR (D₂O) δ 2.86-2.87 (—CH₃, 18H), 3.26 (m, —CH_{eq}, 6H), 3.64-3.75 (—CH₂^{a}, 6H), 4.24-4.36 (—CH₂^{b}, 6H), 5.01 (m, —CH_{eq}, 2H), 5.14 (m, —CH_{eq}, 2H), 5.21 (m, —CH_{eq}, 2H).

Anal. Calcd (%) for C₃₀H₄₂Hf₃N₆O₁₈.12H₂O (1526.34): C, 23.61; H, 4.36; N, 5.51. Found: C, 24.06; H, 4.30; N, 4.83.

IR (cm⁻¹): 513, 526, 535, 550, 567, 578, 606, 630, 648, 675, 696, 722, 819, 838, 914, 930, 1006, 1025, 1092, 1207, 1261, 1323, 1349, 1455, 1477, 1633, 2951, 3445.

MS (ES⁺): m/z (%) 1328.5 (100) {[Hf₃(H₋₃macita)₂]+H+H₂O}⁺, 673.1 (10) {[Hf₃ (H₋₃macita)₂]+2H+2H₂O}²⁺, 1311.2 (8) {[Hf₃(m₋₃tacita)₂]+H}⁺.

The filtrate was sorbed on DOWEX 50 (H⁺-form) which was eluted with water. The fraction from 1.25-1.75 L was lyophilized to get a light yellow solid.

Yield: 75 mg (21%) [Hf₃(H₋₃macita)₂].10H₂O (D₃-symmetric complex as major species).

¹H NMR (D₂O) δ 3.00 (s, —CH₃, 18H), 3.41 (m, —CHₐₓ, 6H), 3.78 (d, —CH₂, J=18 Hz, 6H), 4.47 (d, —CH₂, J=18 Hz, 6H), 5.30 (m, —CH_{eq}, 6H).

¹³C NMR (D₂O) δ 50.2, 62.9, 68.8, 73.7, 183.4.

Anal. Calcd (%) for C₃₀H₄₂Hf₃N₆O₁₈.10H₂O (1490.31): C, 24.18; H, 4.19; N, 5.64. Found: C, 24.36; H, 3.91; N, 4.88.

IR (cm⁻¹): 518, 526, 538, 548, 557, 568, 582, 604, 626, 645, 675, 719, 766, 819, 839, 913, 928, 1004, 1031, 1092, 1129, 1161, 1206, 1260, 1319, 1348, 1449, 1475, 1633, 2891, 3439.

MS (ES⁺): m/z (%) 1329.2 (100) {[Hf₃(H₋₃macita)₂]+H+H₂O}⁺, 673.6 (5) {[Hf₃ (H₋₃macita)₂]+2H+2H₂O}²⁺.

MS (ES⁻): m/z (%) 1354.1 (100) {[Hf₃(H₋₃macita)₂]+HCOO}⁻.

### Example 8

### Na₃[Lu₃(H₋₃macita)₂]

H₃macita.3HCl.H₂O (150 mg, 0.3 mmol) and lutetium(III) chloride hexahydrate (168 mg, 0.4 mmol) were dissolved in water (30 mL). Sodium hydroxide (1 M) was added to adjust the pH to ˜8 and the clear solution was heated to reflux for 2 h. The solvent was removed and the residue was treated with hot ethanol (20 mL). The insoluble salts were filtered off, the filtrate evaporated to dryness and the white solid dried in vacuo.

Yield: 150 mg (67%) Na₃[Lu₃(H₋₃macita)₂].10.5H₂O as a 2:1 mixture (deduced from ¹H NMR) of the C₂- and D₃-symmetric complex species.

¹H NMR (D₂O, pH*=9.5) δ 2.40-2.42 ([3×C₂+D₃]-CHₐₓ, 6H), 2.56-2.61 ([3×C₂+D₃]-CH₃, 18H), 3.02-3.14 ([3×C₂+D₃]-CH₂^{a}, 6H), 3.96-4.00 ([3×C₂+D₃]-CH₂^{b}, 6H), 4.55 (m, [C₂]—CH_{eq}, 1.3H), 4.58-4.59 ([2×C₂+D₃]-CH_{eq}, 4.7H).

¹³C NMR (D₂O, pH*=9.5) δ 45.9 (×2), 46.0 (×2), 60.6, 60.8, 60.9, 61.1, 69.5, 69.6, 69.7, 69.9, 70.07, 70.13, 70.2, 70.4, 185.88, 185.92, 185.97, 186.04.

Anal. Calcd (%) for C₃₀H₄₂Lu₃N₆Na₃O₁₈.10.5H₂O (1557.71): C, 23.13; H, 4.08; N, 5.40; Lu, 33.70. Found: C, 23.49; H, 3.81; N, 5.32; Lu, 33.60.

IR (cm⁻¹): 515, 545, 556, 573, 596, 605, 627, 649, 720, 805, 823, 914, 1006, 1036, 1114, 1147, 1220, 1258, 1326, 1392, 1471, 1581, 2862, 3396.

MS (ES⁺): m/z (%) 707.4 (100) {[Lu₃(H₋₃macita)₂]+5Na}²⁺, 1391.5 (33) {[Lu₃ (H₋₃macita)₂]+4Na}⁺, 1325.5 (7) {[Lu₃(H₋₃macita)₂]+3H+Na}⁺.

MS (ES⁻): m/z (%) 433.5 (100) {[Lu₃(H₋₃macita)₂]}³⁻, 661.4 (37) {[Lu₃(H₋₃macita)₂]+Na}²⁻, 650.5 (35) {[Lu₃(H₋₃macita)₂]+H}²⁻, 1345.6 (23) {[Lu₃(H₋₃macita)₂]+2Na}⁻.

### Example 9

### Na₃[Gd₃(H₋₃macita)₂]

The complex was prepared according to the protocol for the lutetium complex Na₃[Lu₃ (H₋₃macita)₂] using H₃macita.3HCl.H₂O (150 mg, 0.3 mmol) and gadolinium(III) chloride hexahydrate (160 mg, 0.4 mmol) as starting material.

Yield: 150 mg (70%) Na₃[Gd₃(H₋₃macita)₂].7H₂O.EtOH.

Anal. Calcd (%) for C₃₀H₄₂Gd₃N₆Na₃O₁₈.7H₂O.EtOH (1487.58): C, 25.84; H, 4.20; N, 5.65. Found: C, 25.74; H, 4.27; N, 5.60.

IR (cm⁻¹): 517, 543, 556, 566, 581, 624, 634, 718, 799, 817, 911, 961, 1001, 1036, 1111, 1221, 1258, 1326, 1385, 1471, 1575, 2870, 3372.

MS (ES⁺): m/z (%) 1338.1 (100) {[Gd₃(H₋₃macita)₂]+4Na}⁺, 1272.0 (21) {[Gd₃ (H₋₃macita)₂]+3H+Na}⁺.

### Example 10

### Na₃[Ho₃(H₋₃macita)₂]

The complex was prepared from H₃macita.3HCl.H₂O (150 mg, 0.3 mmol) and holmium(III) chloride hexahydrate (164 mg, 0.4 mmol) by following the protocol for the preparation of the lutetium complex Na₃[Lu₃(H₋₃macita)₂].

Yield: 200 mg (91%) Na₃[Ho₃(H₋₃macita)₂].10H₂O.

Anal. Calcd (%) for C₃₀H₄₂Ho₃N₆Na₃O₁₈.10H₂O (1518.60): C, 23.73; H, 4.12; N, 5.53. Found: C, 23.56; H, 4.19; N, 5.40.

IR (cm⁻¹): 518, 528, 543, 550, 584, 598, 620, 641, 672, 720, 802, 819, 911, 961, 1003, 1036, 1113, 1146, 1220, 1256, 1325, 1385, 1471, 1582, 2862, 3319.

MS (ES⁺): m/z (%) 1361.2 (100) {[Ho₃(H₋₃macita)₂]+4Na}⁺, 1295.2 (22) {[Ho₃ (H₋₃macita)₂]+3H+Na}⁺.

### Example 11

### Na₃[Er₃(H₋₃macita)₂]

The complex was prepared according to the protocol for the lutetium complex Na₃[Lu₃(H₋₃macita)₂] using H₃macita.3HCl.H₂O (150 mg, 0.3 mmol) and erbium(III) chloride hexahydrate (165 mg, 0.4 mmol) as starting material.

Yield: 140 mg (63%) Na₃[Er₃(H₋₃macita)₂].11H₂O.

Anal. Calcd (%) for C₃₀H₄₂Er₃N₆Na₃O₁₈.11H₂O (1543.60): C, 23.34; H, 4.18; N, 5.44. Found: C, 23.33; H, 4.04; N, 5.25.

IR (cm⁻¹): 517, 527, 538, 557, 577, 609, 638, 666, 718, 803, 821, 912, 1005, 1036, 1113, 1221, 1258, 1326, 1386, 1471, 1582, 2869, 3355.

MS (ES⁺): m/z (%) 1368.1 (100) {[Er₃(H₋₃macita)₂]+4Na}⁺, 1302.1 (23) {[Er₃ (H₋₃macita)₂]+3H+Na}⁺.

### Example 12

### Na₃[Yb₃(H₋₃macita)₂]

H₃macita.3HCl.H₂O (400 mg, 0.8 mmol) and ytterbium(III) chloride hexahydrate (398 mg, 1.0 mmol) were dissolved in water (30 mL). Sodium hydroxide (1 M) was added to adjust the pH to ˜8 and the clear solution was heated to reflux for 3 h. The solution was desalted via ultra filtration (cellulose acetate membrane, lowest NMWL 500 g/mol, Millipore). The filtrate was evaporated to dryness and the white solid dried in vacuo.

Yield: 320 mg (60%) as Na₃[Yb₃(H₋₃macita)₂].H₂O.

Anal. Calcd (%) for C₃₀H₄₂N₆Na₃O₁₈Yb₃.H₂O (1380.83): C, 26.10; H, 3.21; N, 6.09. Found: C, 26.21; H, 3.50; N, 6.10.

IR (cm⁻¹): 520, 536, 548, 569, 578, 586, 597, 619, 639, 694, 718, 804, 822, 913, 1007, 1035, 1113, 1147, 1257, 1324, 1386, 1470, 1573, 2875, 3356.

MS (ES⁺): m/z (%) 1385.7 (100) {[Yb₃(H₋₃macita)₂]+4Na}⁺, 1364.7 (6) {[Yb₃ (H₋₃macita)₂]+H+3Na}⁺, 1320.7 (4) {[Yb₃(H₋₃macita)₂]+3H+Na}⁺.

MS (ES⁻): m/z (%) 1340.6 (100) {[Yb₃(H₋₃macita)₂]+2Na}⁻, 1318.7 (22) {[Yb₃ (H₋₃macita)₂]+H+Na}⁻, 1295.7 (17) {[Yb₃(H₋₃macita)₂]+2H}⁻.

### Example 13

### Hf₃(H₋₃tacitp)₂

H₃tacitp.3HCl.3H₂O (500 mg, 0.9 mmol) was dissolved in water (20 mL). 1 M sodium hydroxide (8.1 mL, 8.1 mmol) as well as hafnium(IV) chloride (489 mg, 1.5 mmol) dissolved in water (5 mL) were successively added. The pH was adjusted to ˜3 (1 M hydrochloric acid) and the suspension was heated to reflux for 3 days. The solids were filtered off and the filtrate was passed through a mixed bed ionic exchange column (Amberlite MB-6113) which was eluted with water (500 mL). The eluate was lyophilized to get the product as a white solid.

Yield: 320 mg (47%) [Hf₃(H₋₃tacitp)₂].11.5H₂O as a 1:1 mixture (deduced from ¹H NMR and from HPLC) of the C₂- and D₃-symmetric complex species. Single crystals of the composition D₃-[Hf₃(H₋₃tacitp)₂].9H₂O suitable for X-ray analysis were obtained by slow evaporation of a solution of the compound in a water/ethanol mixture.

¹H NMR (D₂O, pH* ˜7) δ 2.51-2.65 ([6×C₂+2×D₃]-CH₂COO, 12H), 3.15-3.18 ([3×C₂+D₃]-CH₂^{a}N, 6H), 3.24-3.32 ([3×C₂+D₃]-CH₂^{b}N, 6H), 3.46 (m, [C₂]—CHₐₓ, 1H), 3.50 (m, [C₂]—CHₐₓ, 1H), 3.53 (m, [D₃]-CHₐₓ, 3H), 3.57 (m, [C₂]—CHₐₓ, 1H), 4.75 (m, [C₂]—CH_{eq}, 1H), 4.90-5.00 ([3×C₂+D₃]-NH₂, 6H), 5.03 ([C₂+D₃]-CH_{eq}, 4H), 5.30 (m, [C₂]—CH_{eq}, 1H).

¹³C NMR (D₂O, pH* ˜7) δ 36.1, 36.19, 36.22, 36.3, 44.8 (×2), 44.85, 44.87, 62.1, 62.15, 62.24, 62.3, 74.7, 76.6, 76.7, 78.4, 182.6 (×2), 182.7 (×2).

Anal. Calcd (%) for C₃₀H₄₂Hf₃N₆O₁₈.11.5H₂O (1517.33): C, 23.75; H, 4.32; N, 5.54. Found: C, 23.69; H, 3.93; N, 5.32.

IR (cm⁻¹): 614, 817, 884, 1010, 1360, 1624, 1984, 2059, 2144, 2167, 3207, 3264, 3424, 3465, 3483, 3729, 3865.

MS (ES⁻): m/z (%) 1355.2 (100) {[Hf₃(H₋₃tacitp)₂]+HCOO}⁻, 1309.2 (15) {[Hf₃(H₋₃tacitp)₂]−H}⁻.

### Crystal Data and Structure Refinement:

| Empirical formula | C₃₀H₆₀Hf₃N₆O₂₇ | |
|---|---|---|
| Formula weight | 1472.31 | |
| Temperature | 123(2) K | |
| Wavelength | 0.71073 Å | |
| Crystal system | Monoclinic | |
| Space group | C2/c | |
| Unit cell dimensions | a = 19.3300(16) Å | α = 90°. |
| | b = 18.2638(16) Å | β = 99.968(6)°. |
| | c = 12.0345(10) Å | γ = 90°. |

| | |
|---|---|
| Volume | 4184.5(6) Å³ |
| Z | 4 |
| Density (calculated) | 2.337 Mg/m³ |
| Absorption coefficient | 7.530 mm⁻¹ |
| F(000) | 2856 |
| Crystal size | 0.25 × 0.18 × 0.04 mm³ |
| Theta range for data collection | 1.55 to 33.36°. |
| Index ranges | −29 <= h <= 28, −28 <= |
| | k <= 28, −16 <= l <= 18 |
| Reflections collected | 56887 |
| Independent reflections | 8089 [R(int) = 0.0401] |
| Completeness to theta = 33.36° | 99.6% |
| Absorption correction | Semi-empirical from equivalents |
| Max. and min. transmission | 0.7527 and 0.2547 |
| Refinement method | Full-matrix least-squares on F² |
| Data/restraints/parameters | 8089/9/339 |
| Goodness-of-fit on F² | 1.018 |
| Final R indices [I > 2sigma(I)] | R₁ = 0.0241, wR₂ = 0.0458 |
| R indices (all data) | R₁ = 0.0340, wR₂ = 0.0485 |
| Largest diff. peak and hole | 2.344 and −1.811 e · Å⁻³ |

Atomic coordinates (×10⁴) and equivalent isotropic displacement parameters (Å²×10³) for sh3129. U(eq) is defined as one third of the trace of the orthogonalized U^{ij} tensor.

| | x | y | z | U (eq) |
|---|---|---|---|---|
| Hf (1) | 5764 (1) | 1918 (1) | 1894 (1) | 11 (1) |
| Hf (2) | 5000 | 3576 (1) | 2500 | 9 (1) |
| C (1) | 6322 (1) | 2846 (1) | 4042 (2) | 12 (1) |
| O (1) | 5945 (1) | 2885 (1) | 2908 (1) | 11 (1) |
| C (2) | 5908 (1) | 3273 (1) | 4795 (2) | 12 (1) |
| N (2) | 5693 (1) | 3975 (1) | 4204 (2) | 12 (1) |
| C (21) | 6292 (1) | 4477 (1) | 4165 (2) | 16 (1) |
| C (22) | 6048 (1) | 5172 (1) | 3534 (2) | 16 (1) |
| C (23) | 5787 (1) | 5070 (1) | 2282 (2) | 15 (1) |
| O (24) | 5614 (1) | 4408 (1) | 1939 (1) | 13 (1) |
| O (25) | 5738 (1) | 5592 (1) | 1637 (2) | 24 (1) |
| C (3) | 5219 (1) | 2899 (1) | 4913 (2) | 12 (1) |
| O (3) | 4746 (1) | 2919 (1) | 3854 (1) | 11 (1) |
| C (4) | 5318 (1) | 2100 (1) | 5262 (2) | 14 (1) |
| N (4) | 4613 (1) | 1754 (1) | 5075 (2) | 15 (1) |
| C (41) | 4142 (2) | 2004 (2) | 5850 (2) | 19 (1) |
| C (42) | 3438 (2) | 1613 (2) | 5601 (2) | 21 (1) |
| C (43) | 2991 (2) | 1839 (1) | 4495 (2) | 18 (1) |
| O (44) | 3316 (1) | 2140 (1) | 3761 (2) | 17 (1) |
| O (45) | 2350 (1) | 1733 (1) | 4328 (2) | 23 (1) |
| C (5) | 5735 (1) | 1681 (1) | 4506 (2) | 13 (1) |
| O (5) | 5325 (1) | 1614 (1) | 3405 (1) | 13 (1) |
| C (6) | 6426 (1) | 2047 (1) | 4380 (2) | 15 (1) |
| N (6) | 6679 (1) | 1675 (1) | 3427 (2) | 14 (1) |
| C (61) | 6909 (2) | 913 (2) | 3704 (2) | 21 (1) |
| C (62) | 7107 (2) | 524 (2) | 2691 (2) | 22 (1) |
| C (63) | 6478 (2) | 343 (2) | 1801 (3) | 29 (1) |
| O (64) | 5961 (1) | 797 (1) | 1632 (2) | 20 (1) |
| O (65A) | 6506 (3) | −174 (3) | 1129 (6) | 41 (2) |
| O (65B) | 6330 (8) | −319 (8) | 1530 (13) | 41 (2) |
| O (1W) | 2321 (1) | 3631 (1) | 1909 (2) | 23 (1) |
| O (2W) | 3134 (1) | 4023 (1) | 3932 (2) | 23 (1) |
| O (3W) | 1907 (1) | 2212 (1) | 2048 (2) | 29 (1) |
| O (4W) | 5000 | 3233 (2) | 7500 | 52 (1) |
| O (5W) | 5314 (4) | −215 (3) | 4153 (6) | 44 (1) |
| O (6W) | 4843 (4) | 329 (4) | 6497 (6) | 49 (2) |

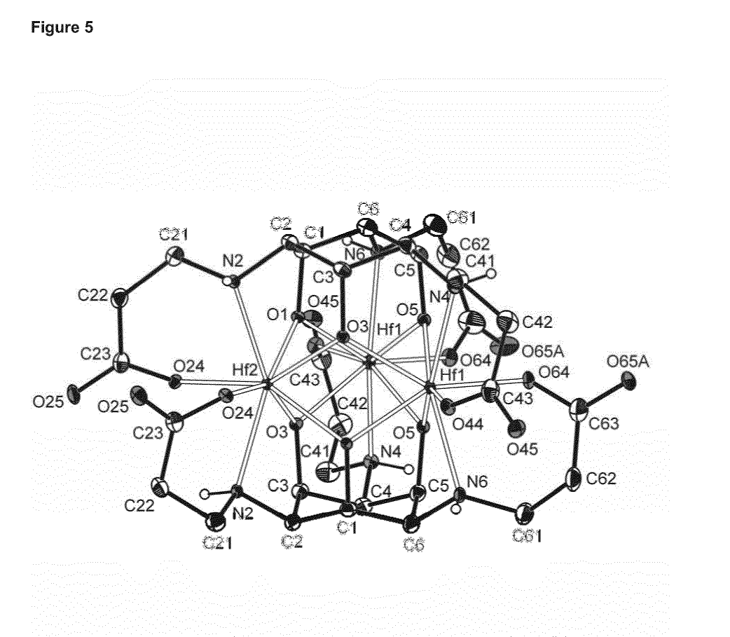

FIG. 5 shows the crystal structure.

### Example 14

### Na₃[Lu₃(H₋₃tacitp)₂]

H₃tacitp.3HCl.3H₂O (100 mg, 0.2 mmol) was dissolved in water (10 mL) and 1.6 eq of lutetium(III) chloride hexahydrate (118 mg dissolved in water, 0.3 mmol) was added. The pH was adjusted to ˜8 (1 M sodium hydroxide). The suspension was stirred at 80° C. for 1 h and filtered afterwards. The solution was desalted via ultra filtration (cellulose acetate membrane, lowest NMWL 500 g/mol, Millipore). The filtrate was evaporated to dryness and the white solid dried in vacuo.

Yield: 70 mg (53%) Na₃[Lu₃(H₋₃tacitp)₂].5.5H₂O as a 1:1 mixture (deduced from ¹H NMR) of the C₂- and D₃-symmetric complex species.

¹H NMR (D₂O, pH* ˜12) δ 2.37-2.51 ([6×C₂+2×D₃]-CH₂COO, 12H), 2.73-2.80 ([3×C₂+D₃]-CH₂^{a}N+[3×C₂+D₃]-CHₐₓ, 12H), 2.97-3.08 ([3×C₂+D₃]-CH₂^{b}N, 6H), 4.19 (m, [C₂]—CH_{eq}, 1H), 4.35 (m, [C₂+D₃]-CH_{eq}, 4H), 4.56 ([C₂]—CH_{eq}, 1 H).

¹³C NMR (D₂O, pH* ˜12) δ 37.8, 37.9, 43.37, 43.41, 43.5, 43.6, 63.8 (×2), 63.9 (×2), 69.2, 72.9, 73.0, 76.3, 171.2, 185.7.

Anal. Calcd (%) for C₃₀H₄₂Lu₃N₆Na₃O₁₈.5.5H₂O (1467.64): C, 24.55; H, 3.64; N, 5.73. Found: C, 24.86; H, 4.02; N, 5.22.

IR (cm⁻¹): 629, 867, 954, 1005, 1138, 1370, 1570, 2024, 2070, 2187, 2357, 3217, 3411, 3668.

MS (ES⁺): m/z (%) 1391.3 (100) {[Lu₃(H₋₃tacitp)₂]+4Na}⁺, 707.3 (73) {[Lu₃ (H₋₃tacitp)₂]+5Na}²⁺, 1369.3 (10) {[Lu₃(H₋₃tacitp)₂]+3Na+H}⁺.

MS (ES⁻): m/z (%) 661.4 (100) {[Lu₃(H₋₃tacitp)₂]+Na}²⁻, 433.4 (50) {[Lu₃(H₋₃tacitp)₂]}³⁻, 650.3 (45) {[Lu₃(H₋₃tacitp)₂]+H}²⁻, 1345.5 (40) {[Lu₃(H₋₃tacitp)₂]+2Na}⁻, 1323.5 (12) {[Lu₃ (H₋₃tacitp)₂]+Na+H}⁻.

### Example 15

### Na₃[Ho₃(H₋₃tacitp)₂]

The complex was prepared according to the protocol for the lutetium complex Na₃[Lu₃ (H₋₃tacitp)₂] using H₃tacitp.3HCl.3H₂O (100 mg, 0.2 mmol) and holmium(III) chloride hexahydrate (109 mg, 0.3 mmol) as starting material.

Yield: 65 mg (49%) Na₃[Ho₃(H₋₃tacitp)₂].8H₂O. Single crystals of the composition D₃-K₃[Ho₃(H₋₃tacitp)₂].14.5H₂O were obtained by slow evaporation of an aqueous solution of the complex (potassium hydroxide used in the synthesis).

Anal. Calcd (%) for C₃₀H₄₂Ho₃N₆Na₃O₁₈.8H₂O (1482.57): C, 24.30; H, 3.94; N, 5.67. Found: C, 24.10; H, 3.70; N, 5.94.

IR (cm⁻¹): 611, 870, 951, 1002, 1103, 1134, 1394, 1556, 3252.

MS (ES⁺): m/z (%) 1361.7 (100) {[Ho₃(H₋₃tacitp)₂]+4Na}⁺, 1339.7 (32) {[Ho₃ (H₋₃tacitp)₂]+3Na+H}⁺.

MS (ES⁻): m/z (%) 1271.7 (100) {[Ho₃(H₋₃tacitp)₂]+2H}⁻, 1293.7 (79) {[Ho₃ (H₋₃tacitp)₂+Na+H]}⁻, 1315.7 (58) {[Ho₃(H₋₃tacitp)₂]+2Na}⁻.

### Crystal Data and Structure Refinement:

| Empirical formula | C₃₀H₇₁Ho₃K₃N₆O_{32.50} | |
|---|---|---|
| Formula weight | 1648.02 | |
| Temperature | 100(2) K | |
| Wavelength | 0.71073 Å | |
| Crystal system | Monoclinic | |
| Space group | P2(1)/c | |
| Unit cell dimensions | a = 16.2094(4) Å | α = 90°. |
| | b = 12.5884(3) Å | β = 91.3130(10)°. |
| | c = 25.2981(7) Å | γ = 90°. |

| | |
|---|---|
| Volume | 5160.7(2) Å³ |
| Z | 4 |
| Density (calculated) | 2.121 Mg/m³ |
| Absorption coefficient | 4.900 mm⁻¹ |
| F(000) | 3244 |
| Crystal size | 0.71 × 0.30 × 0.09 mm³ |
| Theta range for data collection | 1.26 to 35.00°. |
| Index ranges | −26 <= h <= 26, −20 <= |
| | k <= 20, −40 <= l <= 40 |
| Reflections collected | 96864 |
| Independent reflections | 22709 [R(int) = 0.0378] |
| Completeness to theta = 35.00° | 99.9% |
| Absorption correction | Semi-empirical from equivalents |
| Max. and min. transmission | 0.6668 and 0.1286 |
| Refinement method | Full-matrix least-squares on F² |
| Data/restraints/parameters | 22709/29/805 |
| Goodness-of-fit on F² | 1.076 |
| Final R indices [I > 2sigma(I)] | R₁ = 0.0246, wR₂ = 0.0531 |
| R indices (all data) | R₁ = 0.0306, wR₂ = 0.0554 |
| Largest diff. peak and hole | 1.919 and −1.088 e · Å⁻³ |

Atomic coordinates (×10⁴) and equivalent isotropic displacement parameters (Å²×10³) for sh3023a. U(eq) is defined as one third of the trace of the orthogonalized U^{ij} tensor.

| | x | | y | | z | U (eq) |
|---|---|---|---|---|---|---|
| Ho (1) | 1424 | (1) | 2675 | (1) | 6704 (1) | 9 (1) |
| Ho (2) | 3077 | (1) | 4658 | (1) | 6681 (1) | 9 (1) |
| Ho (3) | 2492 | (1) | 3212 | (1) | 5475 (1) | 10 (1) |
| K (1) | 1987 | (1) | 4205 | (1) | 7949 (1) | 16 (1) |
| K (2) | 754 | (1) | 1091 | (1) | 5476 (1) | 19 (1) |
| K (3C) | 4297 | (3) | 5141 | (5) | 5438 (3) | 24 (1) |
| K (3B) | 4458 | (7) | 4995 | (13) | 5477 (6) | 22 (2) |
| K (3A) | 3805 | (1) | 6103 | (1) | 5407 (1) | 26 (1) |
| C (11) | 3312 | (1) | 2243 | (2) | 7103 (1) | 12 (1) |
| O (11) | 2722 | (1) | 3065 | (1) | 7053 (1) | 11 (1) |
| C (12) | 4123 | (1) | 2555 | (2) | 6838 (1) | 13 (1) |
| N (12) | 4319 | (1) | 3645 | (1) | 7035 (1) | 14 (1) |
| C (121) | 5153 | (1) | 4024 | (2) | 6914 (1) | 18 (1) |
| C (122) | 5285 | (1) | 5178 | (2) | 7076 (1) | 21 (1) |
| C (123) | 4859 | (1) | 5999 | (2) | 6719 (1) | 19 (1) |
| O (124) | 5193 | (1) | 6894 | (2) | 6674 (1) | 38 (1) |
| O (125) | 4190 | (1) | 5757 | (1) | 6482 (1) | 17 (1) |
| C (13) | 4052 | (1) | 2576 | (2) | 6230 (1) | 13 (1) |
| O (13) | 3588 | (1) | 3470 | (1) | 6053 (1) | 13 (1) |
| C (14) | 3682 | (1) | 1527 | (2) | 6014 (1) | 13 (1) |
| N (14) | 3510 | (1) | 1687 | (1) | 5439 (1) | 14 (1) |
| C (141) | 3181 | (2) | 722 | (2) | 5176 (1) | 18 (1) |
| C (142) | 3038 | (1) | 918 | (2) | 4586 (1) | 19 (1) |
| C (143) | 2315 | (1) | 1635 | (2) | 4445 (1) | 18 (1) |
| O (144) | 2114 | (1) | 1745 | (2) | 3967 (1) | 29 (1) |
| O (145) | 1927 | (1) | 2094 | (1) | 4816 (1) | 18 (1) |
| C (15) | 2874 | (1) | 1210 | (2) | 6271 (1) | 12 (1) |
| O (15) | 2227 | (1) | 1888 | (1) | 6094 (1) | 12 (1) |
| C (16) | 2964 | (1) | 1200 | (2) | 6875 (1) | 12 (1) |
| N (16) | 2122 | (1) | 1036 | (1) | 7081 (1) | 13 (1) |
| C (161) | 2090 | (1) | 843 | (2) | 7655 (1) | 18 (1) |
| C (162) | 1199 | (1) | 820 | (2) | 7843 (1) | 18 (1) |
| C (163) | 786 | (1) | 1901 | (2) | 7888 (1) | 16 (1) |
| O (164) | 250 | (1) | 2023 | (1) | 8232 (1) | 25 (1) |
| O (165) | 991 | (1) | 2633 | (1) | 7573 (1) | 17 (1) |
| C (21) | 1863 | (1) | 5600 | (2) | 5763 (1) | 11 (1) |
| O (21) | 2543 | (1) | 4901 | (1) | 5839 (1) | 12 (1) |
| C (22) | 1557 | (1) | 6038 | (1) | 6291 (1) | 11 (1) |
| N (22) | 2302 | (1) | 6403 | (1) | 6590 (1) | 13 (1) |
| C (221) | 2100 | (1) | 6994 | (2) | 7074 (1) | 16 (1) |
| C (222) | 2873 | (1) | 7309 | (2) | 7390 (1) | 18 (1) |
| C (223) | 3296 | (1) | 6408 | (2) | 7687 (1) | 16 (1) |
| O (224) | 3707 | (1) | 6634 | (1) | 8096 (1) | 31 (1) |
| O (225) | 3214 | (1) | 5459 | (1) | 7516 (1) | 16 (1) |
| C (23) | 1114 | (1) | 5199 | (2) | 6621 (1) | 12 (1) |
| O (23) | 1684 | (1) | 4448 | (1) | 6828 (1) | 11 (1) |
| C (24) | 424 | (1) | 4651 | (1) | 6297 (1) | 11 (1) |
| N (24) | 131 | (1) | 3769 | (1) | 6633 (1) | 12 (1) |
| C (241) | −634 | (1) | 3245 | (2) | 6446 (1) | 15 (1) |
| C (242) | −863 | (1) | 2331 | (2) | 6807 (1) | 17 (1) |
| C (243) | −351 | (1) | 1332 | (2) | 6743 (1) | 16 (1) |
| O (244) | −661 | (1) | 466 | (1) | 6877 (1) | 31 (1) |
| O (245) | 373 | (1) | 1418 | (1) | 6561 (1) | 16 (1) |
| C (25) | 711 | (1) | 4199 | (2) | 5769 (1) | 12 (1) |
| O (25) | 1210 | (1) | 3290 | (1) | 5846 (1) | 11 (1) |
| C (26) | 1161 | (1) | 5057 | (2) | 5450 (1) | 13 (1) |
| N (26) | 1519 | (1) | 4501 | (1) | 4993 (1) | 14 (1) |
| C (261) | 1791 | (2) | 5222 | (2) | 4574 (1) | 21 (1) |
| C (262) | 2315 | (2) | 4639 | (2) | 4172 (1) | 23 (1) |
| C (263) | 3176 | (2) | 4493 | (2) | 4385 (1) | 29 (1) |
| O (26A) | 3768 | (2) | 5204 | (3) | 4319 (2) | 26 (1) |
| O (26B) | 3691 | (3) | 4859 | (3) | 4069 (2) | 31 (1) |
| O (265) | 3305 | (1) | 3854 | (1) | 4772 (1) | 21 (1) |
| O (1W) | 3469 | (1) | 3191 | (2) | 8342 (1) | 27 (1) |
| O (2W) | 519 | (1) | 3005 | (2) | 4302 (1) | 28 (1) |
| O (3W) | 4422 | (1) | 19 | (2) | 3594 (1) | 31 (1) |
| O (4W) | 1389 | (1) | −823 | (2) | 5842 (1) | 32 (1) |
| O (5W) | −590 | (1) | 1044 | (2) | 3213 (1) | 37 (1) |
| O (6W) | 5208 | (2) | 7170 | (2) | 5497 (1) | 41 (1) |
| O (7W) | 5786 | (2) | 1267 | (2) | 3390 (1) | 49 (1) |
| O (8W) | −746 | (2) | −74 | (2) | 5561 (1) | 37 (1) |
| O (9W) | 4871 | (2) | 4430 | (3) | 5471 (1) | 27 (1) |
| O (10W) | −644 | (2) | 2107 | (2) | 4937 (1) | 36 (1) |
| O (11W) | 2938 | (1) | −1772 | (2) | 5908 (1) | 42 (1) |
| O (12W) | 5282 | (2) | 738 | (3) | 5375 (1) | 54 (1) |
| O (13W) | 1888 | (2) | 8308 | (2) | 4352 (1) | 57 (1) |
| O (14A) | 3212 | (4) | 7064 | (5) | 3925 (2) | 35 (1) |
| O (14B) | 2931 | (4) | 6946 | (5) | 3751 (3) | 39 (1) |
| O (15A) | 3430 | (4) | 7954 | (4) | 4951 (2) | 42 (1) |
| O (15B) | 3751 | (4) | 7586 | (7) | 4940 (3) | 68 (2) |

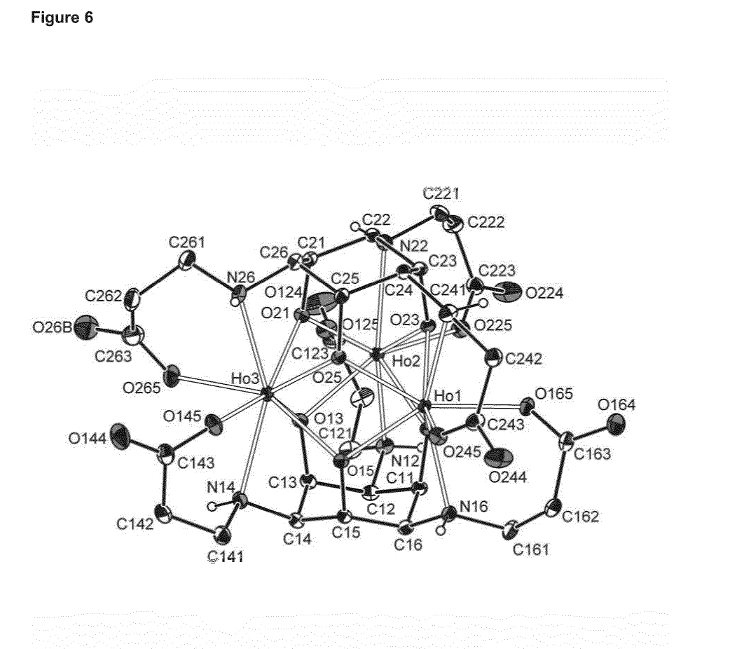

FIG. 6 shows the crystal structure.

### Example 16

### Na₃[Er₃(H₋₃tacitp)₂]

H₃tacitp.3HCl.3H₂O (100 mg, 0.2 mmol) was dissolved in water (10 mL) and 1.6 eq of erbium(III) chloride hexahydrate (110 mg, 0.3 mmol) dissolved in water (10 mL) was added. The pH was adjusted to ˜8 (1 M sodium hydroxide). The suspension was stirred at 80° C. for 1 h and filtered afterwards. The solvent was removed and the residue was treated with hot ethanol (50 mL). The insoluble salts were filtered off, the filtrate evaporated to dryness and the rose solid dried in vacuo.

Yield: 58 mg (40%) Na₃[Er₃(H₋₃tacitp)₂].15H₂O.

Anal. Calcd (%) for C₃₀H₄₂Er₃N₆Na₃O₁₈.15H₂O (1615.66): C, 22.30; H, 4.49; N, 5.20. Found: C, 22.18; H, 4.07; N, 5.24.

IR (cm⁻¹): 606, 626, 655, 875, 952, 1003, 1135, 1397, 1556, 2031, 3431, 3486.

### Example 17

### Na₃[Yb₃(H₋₃tacitp)₂]

The complex was prepared according to the protocol for the erbium complex Na₃[Er₃(H₋₃tacitp)₂] using H₃tacitp.3HCl.3H₂O (100 mg, 0.2 mmol) and ytterbium(III) chloride hexahydrate (112 mg, 0.3 mmol) as starting material.

Yield: 79 mg (54%) Na₃[Yb₃(H₋₃tacitp)₂].13H₂O.

Anal. Calcd (%) for C₃₀H₄₂N₆Na₃O₁₈Yb₃.15H₂O (1633.04): C, 22.06; H, 4.44; N, 5.15. Found: C, 21.95; H, 4.20; N, 5.09.

IR (cm⁻¹): 619, 789, 871, 953, 1002, 1070, 1102, 1135, 1274, 1396, 1557, 2850, 3260.

### Example 18

### Hf₃(H₋₃macitp)₂

H₃macitp.3HCl.4.5H₂O (1.3 g, 2.1 mmol) was dissolved in water (100 mL) and treated with sodium hydroxide (18.7 mL of a 1 M solution, 18.7 mmol). Hafnium (IV) tetrachloride (1.1 g, 3.4 mmol) dissolved in a small amount of water was added and the pH was adjusted to ˜3 (adjusted with 1 M hydrochloric acid). The solution was heated to reflux for 3 days. The white solid was filtered off and the filtrate was passed through a mixed bed ionic exchange column (Amberlite MB-6113) which was eluted with water. The eluate was lyophilized to get the 1.23 g raw product as a white solid which was purified by preparative HPLC.

| Column: | C18 YMC-ODS AQ 10 μm 51 × 200 mm |
|---|---|
| Solvent: | A = H₂O + 0.05% HCOOH |
| | B = acetonitrile |
| Gradient: | 0-2 min 1% B, 2-11 min 1-40% B |
| Flow: | 240 mL/min |
| Temperature: | RT |
| Detection: | 195 nm |
| Rt in min: | 6.98-7.49 |

Yield: 44 mg [Hf₃(H₋₃macitp)₂].xH₂O.

¹H NMR (D₂O) δ 2.48-2.67 (m, 12H), 2.78-2.92 (m, 6H), 2.85 (s, 9H), 2.87 (s, 9H), 2.92-3.03 (m, 6H), 3.61-3.81 (m, 6H), 5.48 (m, 6H).

MS (ES⁻): m/z (%) 1395.5 (100) {[Hf₃(H₋₃macitp)₂]+H}, 1417.4 (50) {[Hf₃(H₋₃macitp)₂]+Na}

MS (ES⁻): m/z (%) 1439.4 (100) {[Hf₃(H₋₃macitp)₂]+HCOO}, 1393.5 (12) {[Hf₃(H₋₃macitp)₂]−H}⁻.

### Example 19

### Na₃[Lu₃(H₋₃macitp)₂]

The complex was prepared according to the protocol for the erbium complex Na₃[Er₃ (H₋₃tacitp)₂] using H₃macitp.3HCl.4.5H₂O (100 mg, 0.2 mmol) and lutetium(III) chloride hexahydrate (100 mg, 0.3 mmol) as starting material.

Yield: 68 mg (56%) Na₃[Lu₃(H₋₃macitp)₂].2.5H₂O.0.5EtOH as a mixture of the C₂- and D₃-symmetric complex species. Single crystals of the composition C₂—K₃[Lu₃ (H₋₃macitp)₂].11H₂O were obtained by slow evaporation of a solution of the complex (potassium hydroxide used in the synthesis) in a water/acetone mixture.

¹H NMR (D₂O) δ 2.07-2.08 ([3×C₂+D₃]-CHₐₓ+[3×C₂+D₃]-CH₂^{a}N, 12H), 2.32-2.36 ([3×C₂+D₃]-CH₂^{a}COO, 6H), 2.49-2.50 ([3×C₂+D₃]-CH₃, 18H), 2.73-2.80 ([3×C₂+D₃]-CH₂^{b}COO, 6H), 3.52-3.60 ([3×C₂+D₃]-CH₂^{b}N, 6H), 4.72-4.83 ([3×C₂+D₃]-CH_{eq}, 6H).

¹³C NMR (D₂O) δ 34.98, 35.01, 35.03, 35.1, 42.59, 42.61, 42.63, 42.7, 51.81, 51.84 (×2), 51.9, 67.2, 68.3 (×2), 69.5, 72.3 (×2), 72.37, 72.42, 185.16, 185.22, 185.25, 185.33.

Anal. Calcd (%) for C₃₆H₅₄Lu₃N₆Na₃O₁₈.2.5H₂O.0.5EtOH (1520.79): C, 29.22; H, 4.11; N, 5.53. Found: C, 29.05; H, 4.15; N, 5.14.

IR (cm⁻¹): 614, 666, 859, 910, 945, 992, 1116, 1147, 1226, 1285, 1325, 1395, 1556, 2025, 2162, 2198, 2816, 3312.

MS (ES⁺): m/z (%) 1475.6 (100) {[Lu₃(H₋₃macitp)₂]+4Na}⁺, 1453.6 (35) {[Lu₃ (H₋₃macitp)₂]+3Na+H}⁺, 1431.6 (20) {[Lu₃(H₋₃macitp)₂]+2Na+2H}⁺.

MS (ES⁻): m/z (%) 703.5 (100) {[Lu₃(H₋₃macitp)₂]+Na}²⁻, 1429.8 (40) {[Lu₃ (H₋₃macitp)₂]+2Na}⁻, 692 (13) {[Lu₃(H₋₃macitp)₂]+H}²⁻, 1407 (13) {[Lu₃(H₋₃macitp)₂]+Na+H}⁻.

### Crystal Data and Structure Refinement:

| Empirical formula | C₃₆H₇₆K₃Lu₃N₆O₂₉ | |
|---|---|---|
| Formula weight | 1699.24 | |
| Temperature | 153(2) K | |
| Wavelength | 0.71073 Å | |
| Crystal system | Orthorhombic | |
| Space group | Pnma | |
| Unit cell dimensions | a = 21. 9991(7) Å | α = 90°. |
| | b = 16.9419(6) Å | β = 90°. |
| | c = 15.0754(6) Å | γ = 90°. |

| | |
|---|---|
| Volume | 5618.7(3) Å³ |
| Z | 4 |
| Density (calculated) | 2.009 Mg/m³ |
| Absorption coefficient | 5.544 mm⁻¹ |
| F(000) | 3344 |
| Crystal size | 0.59 × 0.19 × 0.09 mm³ |
| Theta range for data collection | 1.64 to 28.37°. |
| Index ranges | −29 <= h <= 17, −22 <= |
| | k <= 22, −20 <= l <= 19 |
| Reflections collected | 29222 |
| Independent reflections | 7232 [R(int) = 0.0358] |
| Completeness to theta = 28.37° | 99.6% |
| Absorption correction | Semi-empirical from equivalents |
| Max. and min. transmission | 0.6353 and 0.1384 |
| Refinement method | Full-matrix least-squares on F² |
| Data/restraints/parameters | 7232/0/346 |
| Goodness-of-fit on F² | 1.067 |
| Final R indices [I > 2sigma(I)] | R₁ = 0.0543, wR₂ = 0.1494 |
| R indices (all data) | R₁ = 0.0801, wR₂ = 0.1597 |
| Largest diff. peak and hole | 1.937 and −1.873 e · Å⁻³ |

Atomic coordinates (×10⁴) and equivalent isotropic displacement parameters (Å²×10³) for sh3050. U(eq) is defined as one third of the trace of the orthogonalized U^{ij} tensor.

| | x | | y | | z | | U (eq) |
|---|---|---|---|---|---|---|---|
| Lu (1) | 1982 | (1) | 1437 | (1) | 439 | (1) | 34 (1) |
| Lu (2) | 1276 | (1) | 2500 | | 2237 | (1) | 37 (1) |
| K (1) | 1281 | (2) | 348 | (2) | 2273 | (2) | 81 (1) |
| K (2) | 2663 | (2) | 2500 | | −1519 | (2) | 50 (1) |
| N (1) | 1103 | (4) | 1063 | (5) | −608 | (5) | 49 (2) |
| N (2) | 133 | (6) | 2500 | | 1876 | (8) | 69 (4) |
| N (3) | 3027 | (4) | 1061 | (5) | 1051 | (5) | 45 (2) |
| N (4) | 2063 | (5) | 2500 | | 3491 | (6) | 40 (2) |
| O (1) | 1639 | (4) | 2500 | | −336 | (5) | 37 (2) |
| O (2) | 1067 | (3) | 1637 | (4) | 1119 | (4) | 42 (1) |
| O (3) | 2606 | (4) | 2500 | | 477 | (5) | 34 (2) |
| O (4) | 2038 | (3) | 1633 | (3) | 1936 | (4) | 35 (1) |
| C (1) | 1038 | (6) | 2500 | | −666 | (8) | 38 (3) |
| C (2) | 696 | (4) | 1755 | (6) | −389 | (6) | 47 (2) |
| C (3) | 538 | (4) | 1745 | (7) | 589 | (6) | 47 (2) |
| C (4) | 203 | (7) | 2500 | | 854 | (10) | 58 (4) |
| C (5) | 1255 | (6) | 1047 | (7) | −1561 | (7) | 65 (3) |
| C (6) | 789 | (6) | 286 | (6) | −388 | (8) | 72 (4) |
| C (8) | 3104 | (5) | 2500 | | 1074 | (8) | 38 (3) |
| C (9) | 3118 | (4) | 1768 | (5) | 1635 | (6) | 40 (2) |
| C (10) | 2610 | (4) | 1757 | (5) | 2329 | (5) | 37 (2) |
| C (11) | 2616 | (6) | 2500 | | 2920 | (7) | 35 (3) |
| C (12) | 3510 | (5) | 1027 | (7) | 354 | (7) | 54 (3) |
| C (13) | 3087 | (5) | 329 | (6) | 1574 | (7) | 62 (3) |
| C (14) | 2069 | (5) | 1790 | (7) | 4063 | (6) | 52 (3) |
| C (17) | −196 | (6) | 1736 | (11) | 2136 | (8) | 98 (5) |
| C (15) | 1662 | (6) | −494 | (6) | 361 | (6) | 63 (3) |
| C (16) | 2894 | (6) | 558 | (6) | −1000 | (7) | 59 (3) |
| O (5) | 2492 | (3) | 1043 | (4) | −776 | (4) | 49 (2) |
| O (8) | 2898 | (4) | 195 | (5) | −1709 | (5) | 77 (2) |
| O (9) | 898 | (3) | 1586 | (5) | 3184 | (4) | 64 (2) |
| O (10) | 1836 | (3) | 141 | (4) | 734 | (4) | 54 (2) |
| O (11) | 411 | (5) | 1296 | (7) | 4416 | (6) | 95 (3) |
| O (12) | 1788 | (5) | −1158 | (5) | 614 | (6) | 82 (3) |
| C (18) | 1219 | (7) | −409 | (7) | −451 | (8) | 84 (4) |
| C (19) | 3411 | (6) | 376 | (8) | −353 | (7) | 77 (4) |
| C (23) | 1423 | (11) | 1796 | (12) | 4609 | (12) | 56 (6) |
| C (26) | 865 | (13) | 1493 | (12) | 4058 | (13) | 60 (6) |
| C (25) | −191 | (11) | 1590 | (20) | 3160 | (17) | 95 (10) |
| C (27) | 396 | (12) | 1509 | (17) | 3625 | (18) | 78 (8) |
| O (2W) | 3896 | (8) | 2500 | | −1452 | (12) | 149 (8) |
| O (1W) | 2562 | (5) | 1432 | (5) | −2903 | (6) | 89 (3) |

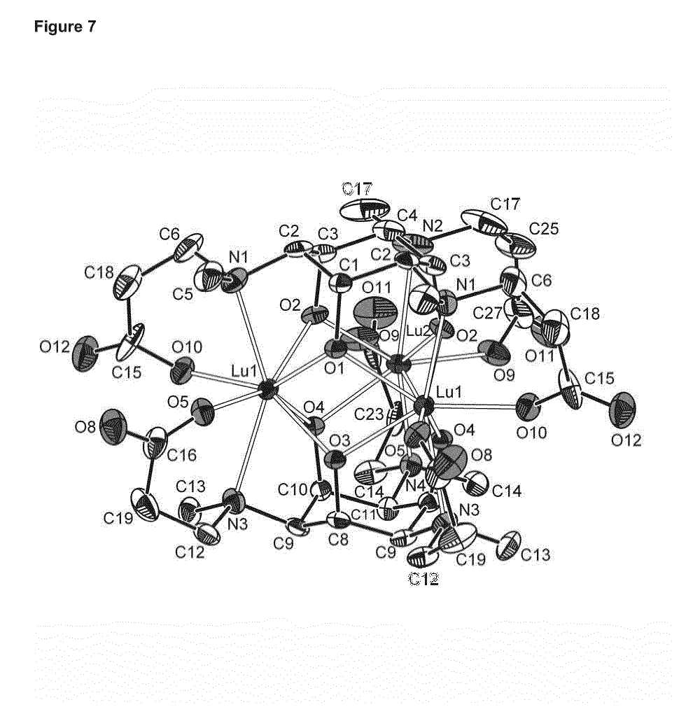

FIG. 7 shows the crystal structure.

### Example 20

Na₃[Gd₃(H₋₃macitp)₂]

The complex was prepared from H₃macitp.3HCl.4.5H₂O (100 mg, 0.2 mmol) and gadolinium(III) chloride hexahydrate (95 mg, 0.3 mmol) by following the protocol for the preparation of the erbium complex Na₃[Er₃(H₋₃tacitp)₂].

Yield: 67 mg (52%) Na₃[Gd₃(H₋₃macitp)₂].11H₂O.

Anal. Calcd (%) for C₃₆H₅₄Gd₃N₆Na₃O₁₈.11H₂O (1597.73): C, 27.06; H, 4.80; N, 5.26. Found: C, 27.03; H, 4.95; N, 5.28.

IR (cm⁻¹): 600, 806, 856, 903, 942, 971, 992, 1024, 1114, 1146, 1285, 1324, 1394, 1474, 1567, 2808, 3323.

MS (ES⁺): m/z (%) 1423.3 (100) {[Gd₃(H₋₃macitp)₂]+4Na}⁺.

### Example 21

### Na₃[Ho₃(H₋₃macitp)₂]

The complex was prepared according to the protocol for the erbium complex Na₃[Er₃ (H₋₃tacitp)₂] using H₃macitp.3HCl.4.5H₂O (100 mg, 0.2 mmol) and holmium(III) chloride hexahydrate (97 mg, 0.3 mmol) as starting material.

Yield: 72 mg (54%) Na₃[Ho₃(H₋₃macitp)₂].13H₂O.

Anal. Calcd (%) for C₃₆H₅₄Ho₃N₆Na₃O₁₈.13H₂O (1656.80): C, 26.10; H, 4.87; N, 5.07. Found: C, 26.05; H, 4.72; N, 5.01.

IR (cm⁻¹): 613, 857, 906, 944, 992, 1026, 1114, 1147, 1285, 1325, 1396, 1568, 2809, 3338.

MS (ES⁺): m/z (%) 1445.9 (100) {[Ho₃(H₋₃macitp)₂]+4Na}⁺.

MS (ES⁻): m/z (%) 1377.9 (100) {[Ho₃(H₋₃macitp)₂]+Na+H}⁻, 1399.7 (90) {[Ho₃ (H₋₃macitp)₂]+2Na}⁻, 1355.9 (77) {[Ho₃(H₋₃macitp)₂]+2H}⁻.

### Example 22

### Na₃[Er₃(H₋₃macitp)₂]

The complex was prepared from H₃macitp.3HCl.4.5H₂O (100 mg, 0.2 mmol) and erbium(III) chloride hexahydrate (98 mg, 0.3 mmol) by following the protocol for the preparation of the erbium complex Na₃[Er₃(H₋₃tacitp)₂].

Yield: 78 mg (58%) Na₃[Er₃(H₋₃macitp)₂].13.5H₂O. Single crystals of the composition C₂—K₃[Er₃(H₋₃macitp)₂].6.5H₂O were obtained by slow evaporation of an aqueous solution of the complex (potassium hydroxide used in the synthesis).

Anal. Calcd (%) for C₃₆H₆₄Er₃N₆Na₃O₁₈.13.5H₂O (1672.80): C, 25.85; H, 4.88; N, 5.02. Found: C, 25.87; H, 5.26; N, 5.17.

IR (cm⁻¹): 613, 857, 907, 944, 992, 1114, 1324, 1394, 1575, 3258.

MS (ES⁺): m/z (%) 1452.3 (100) {[Er₃(H₋₃macitp)₂]+4Na}⁺.

### Crystal Data and Structure Refinement:

| Empirical formula | C₃₆H₆₇Er₃K₃N₆O_{24.50} | |
|---|---|---|
| Formula weight | 1595.04 | |
| Temperature | 200(2) K | |
| Wavelength | 0.71073 Å | |
| Crystal system | Orthorhombic | |
| Space group | Pnma | |
| Unit cell dimensions | a = 22.481(7) Å | α = 90°. |
| | b = 17.041(6) Å | β = 90°. |
| | c = 15.213(4) Å | γ = 90°. |

| | |
|---|---|
| Volume | 5828(3) Å³ |
| Z | 4 |
| Density (calculated) | 1.818 Mg/m³ |
| Absorption coefficient | 4.572 mm⁻¹ |
| F(000) | 3128 |
| Crystal size | 0.49 × 0.29 × 0.08 mm³ |
| Theta range for data collection | 2.55 to 28.14°. |
| Index ranges | −29 <= h <= 29, −22 <= |
| | k <= 22, −19 <= l <= 20 |
| Reflections collected | 52328 |
| Independent reflections | 7222 [R(int) = 0.1223] |
| Completeness to theta = 28.14° | 97.9% |
| Absorption correction | Numerical |
| Max. and min. transmission | 0.7112 and 0.2128 |
| Refinement method | Full-matrix least-squares on F² |
| Data/restraints/parameters | 7222/0/379 |
| Goodness-of-fit on F² | 1.064 |
| Final R indices [I > 2sigma(I)] | R₁ = 0.0678, wR₂ = 0.1676 |
| R indices (all data) | R₁ = 0.0999, wR₂ = 0.1826 |
| Largest diff. peak and hole | 2.319 and −2.617 e · Å⁻³ |

Atomic coordinates (×10⁴) and equivalent isotropic displacement parameters (Å²×10³). U(eq) is defined as one third of the trace of the orthogonalized U^{ij} tensor.

| | x | | y | | z | | U (eq) |
|---|---|---|---|---|---|---|---|
| Er (1) | 1974 | (1) | 1432 | (1) | 480 | (1) | 41 (1) |
| Er (2) | 1252 | (1) | 2500 | | 2276 | (1) | 43 (1) |
| K (1) | 1251 | (2) | 323 | (2) | 2322 | (2) | 84 (1) |
| K (2) | 2655 | (2) | 2500 | | −1488 | (2) | 62 (1) |
| N (1) | 1102 | (5) | 1053 | (6) | −576 | (6) | 57 (2) |
| N (2) | 116 | (6) | 2500 | | 1844 | (9) | 69 (4) |
| N (3) | 3005 | (4) | 1056 | (6) | 1118 | (5) | 54 (2) |
| N (4) | 2035 | (6) | 2500 | | 3542 | (7) | 48 (3) |
| O (1) | 1634 | (4) | 2500 | | −296 | (6) | 43 (2) |
| O (2) | 1052 | (3) | 1645 | (5) | 1142 | (4) | 50 (2) |
| O (3) | 2594 | (4) | 2500 | | 542 | (6) | 40 (2) |
| O (4) | 2016 | (3) | 1642 | (4) | 1985 | (4) | 41 (1) |
| C (1) | 1046 | (7) | 2500 | | −634 | (10) | 48 (3) |
| C (2) | 712 | (5) | 1750 | (8) | −359 | (6) | 53 (3) |
| C (3) | 548 | (5) | 1742 | (9) | 599 | (7) | 60 (3) |
| C (4) | 215 | (8) | 2500 | | 855 | (10) | 70 (5) |
| C (5) | 1248 | (6) | 1052 | (8) | −1532 | (7) | 66 (3) |
| C (6) | 807 | (7) | 312 | (9) | −349 | (9) | 81 (4) |
| C (8) | 3085 | (6) | 2500 | | 1130 | (9) | 45 (3) |
| C (9) | 3085 | (5) | 1761 | (7) | 1702 | (7) | 52 (3) |
| C (10) | 2579 | (5) | 1747 | (7) | 2390 | (6) | 46 (2) |
| C (11) | 2585 | (6) | 2500 | | 2973 | (7) | 39 (3) |
| C (12) | 3490 | (6) | 1022 | (10) | 441 | (8) | 71 (4) |
| C (13) | 3054 | (7) | 343 | (7) | 1668 | (8) | 71 (4) |
| C (14) | 2029 | (6) | 1791 | (8) | 4109 | (7) | 62 (3) |
| C (17) | −206 | (6) | 1786 | (13) | 2130 | (9) | 97 (6) |
| C (15) | 1651 | (7) | −487 | (7) | 356 | (7) | 64 (3) |
| C (16) | 2908 | (6) | 563 | (8) | −936 | (8) | 67 (3) |
| O (5) | 2505 | (4) | 1054 | (5) | −734 | (5) | 59 (2) |
| O (8) | 2932 | (6) | 212 | (7) | −1641 | (6) | 95 (3) |
| O (9) | 862 | (4) | 1579 | (6) | 3232 | (5) | 71 (3) |
| O (10) | 1825 | (4) | 125 | (5) | 748 | (5) | 66 (2) |
| O (11) | 379 | (7) | 1316 | (9) | 4435 | (7) | 118 (5) |
| O (12) | 1786 | (6) | −1149 | (6) | 592 | (7) | 100 (4) |
| C (18) | 1226 | (9) | −411 | (9) | −401 | (9) | 88 (5) |
| C (19) | 3383 | (8) | 374 | (11) | −262 | (9) | 94 (5) |
| C (23) | 1413 | (12) | 1793 | (16) | 4617 | (12) | 64 (7) |
| C (26) | 884 | (15) | 1509 | (17) | 4075 | (15) | 74 (8) |
| C (25) | −208 | (12) | 1630 | (30) | 3100 | (20) | 108 (13) |
| C (27) | 382 | (14) | 1530 | (19) | 3588 | (15) | 78 (9) |
| O (1W) | 1695 | (11) | 2500 | | −3118 | (14) | 153 (9) |
| O (2W) | 3866 | (10) | 2500 | | −1448 | (14) | 179 (12) |
| O (3W) | 449 | (13) | 2500 | | 7000 | (16) | 127 (11) |
| O (4W) | −264 | (11) | 2500 | | 5357 | (15) | 112 (9) |
| O (5W) | 1110 | (10) | −2500 | | 393 | (16) | 89 (7) |
| O (6W) | 4455 | (19) | 2150 | (20) | 6920 | (30) | 86 (12) |
| O (7W) | 2585 | (7) | 1422 | (7) | −2878 | (8) | 115 (5) |

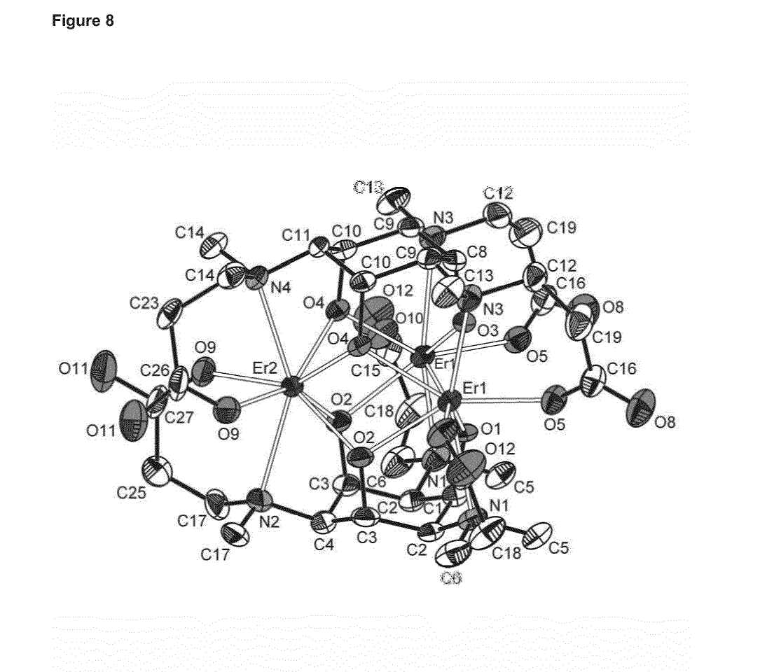

FIG. 8 shows the crystal structure.

### Example 23

### Na₃[Yb₃(H₋₃macitp)₂]

The complex was prepared according to the protocol for the erbium complex Na₃[Er₃ (H₋₃tacitp)₂] using H₃macitp.3HCl.4.5H₂O (100 mg, 0.2 mmol) and ytterbium(III) chloride hexahydrate (99 mg, 0.3 mmol) as starting material.

Yield: 94 mg (72%) Na₃[Yb₃(H₋₃macitp)₂].11H₂O.

Anal. Calcd (%) for C₃₆H₅₄N₆Na₃O₁₈Yb₃.11H₂O (1645.14): C, 26.28; H, 4.66; N, 5.11. Found: C, 26.37; H, 4.64; N, 4.97.

IR (cm⁻¹): 615, 859, 908, 945, 1115, 1324, 1394, 1568, 3296.

MS (ES⁺): m/z (%) 1469.3 (100) {[Yb₃(H₋₃macitp)₂]+4Na}⁺.
1 Ghisletta, M.; Jalett, H.-P.; Gerfin, T.; Gramlich, V.; Hegetschweiler, K. Helv. Chim. Acta 1992, 75, 2233.
2 Bartholomä, M.; Gisbrecht, S.; Stucky, S.; Neis, C.; Morgenstern, B.; Hegetschweiler, K. Chem. Eur. J. 2010, 16, 3326.
3 a) Sheldrick, G. M. SHELXS-97, Program for Crystal Structure Solution, Göttingen, 1990; b) Sheldrick, G. M. SHELXL-97, Program for Crystal Structure Refinement, Göttingen, 1997.
4 Spek, A. L. PLATON, A Multipurpose Crystallographic Tool, Utrecht University, Utrecht, The Netherlands, 2011; see also: Spek, A. L. Acta. Cryst. 2009, D65, 148.

### Example 24

### Stability of Bis Azainositol Heavy Metal Complexes

The stability of bis azainositol heavy metal complexes was determined in aqueous, buffered solution at pH 7.4. The solution containing 5 mmol/L of the compound in a tightly sealed vessel was heated to 121° C. for 45 min in a steam autoclave. The metal concentration of the solution was determined by ICP-OES before and after heat treatment. The integrity of the compound was determined by HPLC analysis before and after heat treatment. Absolute stability was calculated as the ratio of the peak area of the compound after and before the heat treatment multiplied with the ratio of the metal concentration of the solution after and before heat treatment.

### HPLC System:

Column: Reversed phase C18.
Solvent A1: 1 mM hexylamine+1 mM bis-tris pH 6.5
Solvent A2: 0.5 mM tetrabutylammonium phosphate pH 6
The use of solvent A1 to A2 is detailed in the table below.
Solvent B: methanol, HPLC grade
Gradient: gradients starting from 100% A and 0% B were used. Details are given in the table.
Flow: 1 mL/min
Detector D1: element specific detection by ICP-OES running at the most sensitive emission wavelength of the respective complexed metal.
Detector D2: element specific detection by ICP-MS running at the most abundant isotope of the respective complexed metal.

| Example | | Chromatographic conditions | | |
|---|---|---|---|---|
| No | Stability | Solvent A | Gradient | Detector |
| 1 | 102% | A1 | 0-80% B in 15 min | D1 |
| 2 | 100% | A1 | 0-80% B in 15 min | D1 |
| 4 | 100% | A1 | 0-100% B in 10 min | D1 |
| 5 | 85% | A1 | 0-100% B in 10 min | D1 |
| 6 | 99% | A1 | 0-60% B in 9 min | D1 |
| 8 | 100% | A1 | 0-80% B in 15 min | D1 |
| 9 | 100% | A1 | 0-60% B in 9 min | D1 |
| 10 | 100% | A1 | 0-60% B in 9 min | D1 |
| 11 | 100% | A1 | 0-60% B in 9 min | D1 |
| 12 | 100% | A2 | 0-60% B in 10 min | D2 |
| 13 | 101% | A2 | 0-95% B in 10 min | D2 |
| 14 | 98% | A1 | 0-80% B in 15 min | D1 |
| 15 | 88% | A2 | 0-60% B in 10 min | D2 |
| 18 | 100% | A2 | 0-95% B in 10 min | D2 |
| 19 | 90% | A1 | 0-80% B in 15 min | D1 |
| 20 | 101% | A1 | 0-60% B in 9 min | D1 |
| 21 | 100% | A2 | 0-60% B in 10 min | D2 |
| 22 | 100% | A1 | 0-60% B in 9 min | D1 |
| 23 | 96% | A1 | 0-60% B in 9 min | D1 |

### Example 25

### Preclinical X-Ray Imaging

To demonstrate the efficacy of the X-ray diagnostic agent a preclinical animal investigation was performed using X-ray computed tomography (CT). The study was performed on a clinical CT unit (Sensation 64, Siemens Medical Solutions, Erlangen, Germany) with an anaesthetized rat. The compound described in example 2 was used as X-ray diagnostic agents in order to perform contrast enhanced CT imaging.

The study was performed on a healthy Han-Wistar rat. Initial anaesthesia was induced by inhalation of 4% Isoflurane (Baxter Deutschland GmbH, Unterschleiβheim, Germany) and maintained by 1.5% Isoflurane. The X-ray diagnostic agent (Example 2) at a concentration of 149 mg Lu/mL was administered intravenously via the tail vein by the help of a dedicated injection pump (flow rate=0.6 mL/s). A dosage of 200 mg Lu per kg body weight was used. In order to simulate a clinical condition the rat was placed within a tissue equivalent phantom (QRM, Möhrendorf, Germany) that mimics the human abdomen in respect of X-ray absorption. Thus comparable conditions to a situation in humans were ensured regarding X-ray scattering and X-ray beam hardening.

An X-ray projection image (topogram) was acquired to adjust the measurement range to the thoracal region of the animal. The subsequent contrast enhanced measurement was done with following CT parameter settings: X-ray tube voltage=120 kV, mAs-product=160 mAs, tube rotational time=0.5 s, slice thickness=2.4 mm, measurement time=20 s. Imaging was performed without patient table feed resulting in a dynamic imaging of the thoracal region with a temporal resolution of 0.35 s. This allows the sampling of the diagnostic agent bolus during its passage through the vascular system and the heart. The CT measurement was started is prior to contrast agent administration.

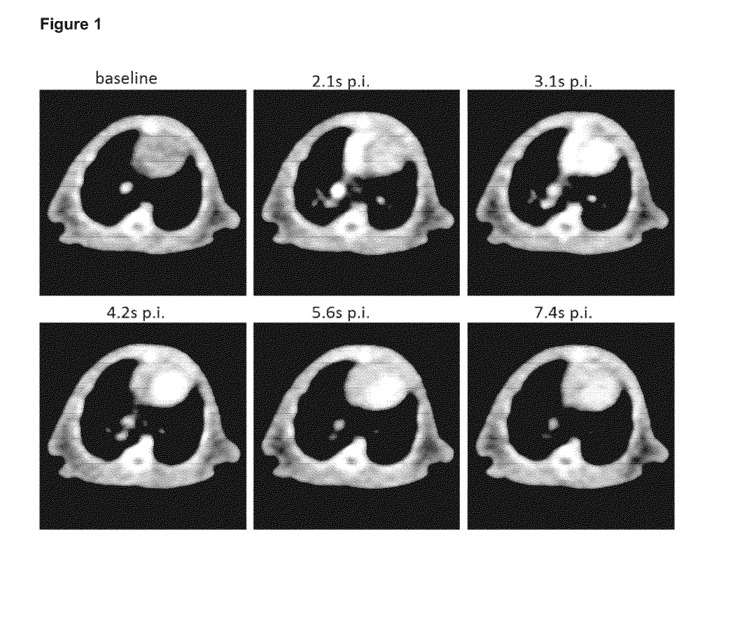

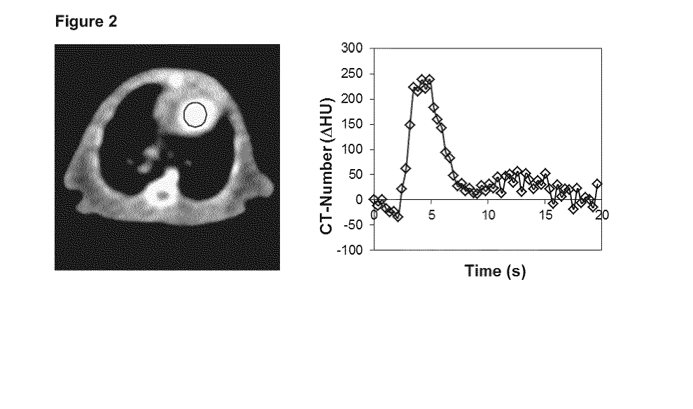

The signal change caused by the diagnostic agent is shown in FIG. 1. The signal time course in the heart and major blood vessels are visualized on representative images: The native baseline image showed an intrinsically high CT signal of the skeleton a medium signal for tissue and low signal for the lung. During the passage of the diagnostic agent a strong signal increase was observed for the blood vessels and heart chambers. The signal-time course in the left heart chamber was quantified by a region of interest analysis. Therefore an identical circular region covering the left heart chamber was drawn on the images. The mean signal value for each time point was normalized to the baseline image resulting in a signal-change time curve (FIG. 2). The high CT-signal during the passage of the diagnostic agent (i.e. between 3-6 s on FIG. 2) demonstrates the highly effective X-ray attenuation of the X-ray diagnostic agent.

### Example 26

### Excretion of [Hf₃(H₋₃tacitp)₂](Example 13) in Rats

An aqueous solution of [Hf₃(H₋₃tacitp)₂](in 10 mM trometamol buffer, pH 7.4, 60 mg Hf/mL) was injected in the tail vein of 3 rats (ca. 100 g) at a dose of 150 mg Hf/kg. Urine samples were collected at the following time intervals: 0-0.5, 0.5-1, 1-3, 3-6, 6-24 h and then daily until day 7. Faeces was collected daily until day 7. On day 7 the animals were sacrificed and the following organs were excised: liver, kidneys, spleen, heart, lung, brain, lymph nodes, muscle, gut, duodenum, skin, bone marrow, bone. The remaining body was freeze dried and ground to obtain a fine powder.

The Hafnium concentration in all specimen was determined after digestion in oxidizing solution (nitric acid and hydrogen peroxide) at elevated pressure and temperature. The measurement of Hafnium was performed by ICP-MS.

After 1 d 96% and after 7 d 97% of the injected Hafnium was excreted via the urine. About 1.3% was found in faeces after 7 d (cumulative data).

In all organs and the carcass together only 0.33% of the injected Hafnium was found after 7 d. The majority of the remaining Hafnium was found in the kidney, the excretion organ. Non of the other organs contained more than 0.01% of the injected dose/g organ (wet weight).

These data indicate fast renal elimination and very low body retention of [Hf₃(H₋₃tacitp)₂] after intravenous administration in rats.

### Example 27

### Pharmacokinetics of [Hf₃(H₋₃tacitp)₂](Example 13) in Rats

An aqueous solution of [Hf₃(H₋₃tacitp)₂](in 10 mM trometamol buffer, pH 7.4, 60 mg Hf/mL) was injected in the tail vein of 3 rats (ca. 250 g) at a dose of 150 mg Hf/kg. Blood samples were collected via a catheter from the arteria carotis at the following times: 1, 2, 5, 10, 15, 30, 60, 90, 120, 240, 360 and 1440 min after injection.

The Hafnium concentration in all blood samples was determined after digestion in oxidizing solution (nitric acid and hydrogen peroxide) at elevated pressure and temperature. The measurement of Hafnium was performed by ICP-MS.

The pharmacokinetic parameters were obtained for each animal by fitting the blood concentrations to a 3-compartment model, using the software WinNonlin.

The third compartment contributed less than 4% to the Area-under-the-curve and was therefore neglected. For the elimination phase the blood half live was 22.6±3.1 min, the volume of distribution was 0.31±0.01 I/kg and total plasma clearance was 10±0.6 mL/min/kg.

These data indicate that [Hf₃(H₋₃tacitp)₂] has pharmacokinetic profile comparable to well established trisiodinated contrast agents.

### Example 28

### Tolerability of Na₃[Lu₃(H₋₃tacita)₂](Example 2) in Mice

An aqueous solution of Na₃[Lu₃(H₋₃tacita)₂](in 10 mM trometamol buffer, pH 7.4, 148 mg Lu/mL) was injected in the tail vein of 1-3 mice for each dose group (22-25 g) at increasing doses ranging from 1000 to 3000 mg Lu/kg. The behaviour of the animals and the survival after 7 d was recorded.

At 1000, 2000 and 2500 mg Lu/kg all animals survived. At 3000 mg Lu/kg 2 of 3 animals died.

1. A trinuclear heavy metal complex comprising two hexadentate azainositol tricarboxylic acid ligands.

2. The trinuclear heavy metal complex, of claim 1, of the general formula (I), 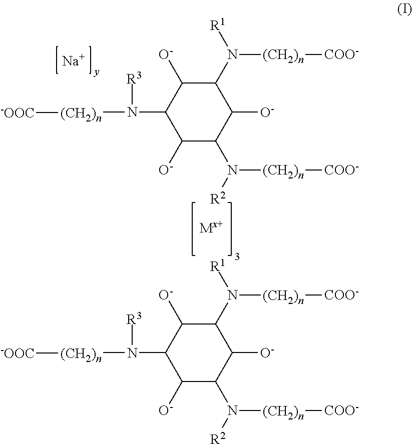
wherein
the substituents at the cyclo hexyl ring exhibit an all-cis configuration;
M is Lanthanum, Cerium, Praseodymium, Neodymium, Samarium, Europium, Gadolinium, Terbium, Dysprosium, Holmium, Erbium, Thulium, Ytterbium, Lutetium, Hafnium or Bismuth;
R¹, R² and R³ are independently selected from H or methyl;
n is 1 or 2;
x is 3 or 4 and
y is 0 or 3;
with the proviso that (3 times x)+y is 12;
or a protonated species or deprotonated species of said complex, an isomeric form of said complex, a pharmaceutically acceptable salt of said complex or a hydrate thereof.

3. The trinuclear heavy metal complex of claim 2,
wherein
M is Gadolinium, Terbium, Dysprosium, Holmium, Erbium, Thulium, Ytterbium, Lutetium, Hafnium or Bismuth;
or a protonated species or deprotonated species of said complex, an isomeric form of said complex, a pharmaceutically acceptable salt of said complex or a hydrate thereof.

4. The trinuclear heavy metal complex of claim 2,
wherein
M is Hafnium;
or a protonated species or deprotonated species of said complex, an isomeric form of said complex, a pharmaceutically acceptable salt of said complex or a hydrate thereof.

5. The trinuclear heavy metal complex of claim 2,
wherein
R¹, R² and R³ are methyl;
or a protonated species or deprotonated species of said complex, an isomeric form of said complex, a pharmaceutically acceptable salt of said complex or a hydrate thereof.

6. The trinuclear heavy metal complex of claim 2,
wherein
M is Hafnium and
R¹, R² and R³ are methyl;
or a protonated species or deprotonated species of said complex, an isomeric form of said complex, a pharmaceutically acceptable salt of said complex or a hydrate thereof.

7. The trinuclear heavy metal complex of claim 2, selected from the group consisting of:
[Hf₃(H₋₃tacita)₂]=Bis {μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}trihafnium(IV),
Na₃[Lu₃(H₋₃tacita)₂]=Trisodium bis {μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}trilutetate(III),
Na₃[Gd₃(H₋₃tacita)₂]=Trisodium bis {μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}trigadolinate(III),
Na₃[Ho₃(H₋₃tacita)₂]=Trisodium bis {μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}triholmate(III),
Na₃[Er₃(H₋₃tacita)₂]=Trisodium bis {μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}trierbate(III)
Na₃[Yb₃(H₋₃tacita)₂]=Trisodium bis {μ₃-[(all-cis)-2-{[(carboxy-1κO)methyl]amino-1κN}-4-{[(carboxy-2κO)methyl]amino-2κN}-6-{[(carboxy-3κO)methyl]amino-3κN}cyclohexane-1,3,5-triolate-1κ²O¹,O³:2κ²O³,O⁵:3κ²O¹,O⁵]}triytterbate(III),
[Hf₃(H₋₃macita)₂]=Bis {μ₃-[(all-cis)-2-{[(carboxy-1κO)methyl](methyl)amino-1κN}-4-{[(carboxy-2κO)methyl](methyl)amino-2κN}-6-{[(carboxy-3κO)methyl](methyl)amino-3κN}cyclohexane-1,3,5-triolate-1κ²O¹,O³:2κ²O³,O⁵:3κ²O¹,O⁵]}trihafnium(IV),
Na₃[Lu₃(H₋₃macita)₂]=Trisodium bis {μ₃-[(all-cis)-2-{[(carboxy-1κO)methyl](methyl)-amino-1κN}-4-{[(carboxy-2κO)methyl](methyl)amino-2κN}-6-{[(carboxy-3κO)methyl]-(methyl)amino-3κN}cyclohexane-1,3,5-triolate-1κ²O¹,O³:2κ²O³,O⁵:3κ²O¹,O⁵]}trilutetate(III),
Na₃[Gd₃(H₋₃macita)₂]=Trisodium bis {μ₃-[(all-cis)-2-{[(carboxy-1κO)methyl](methyl)-amino-1κN}-4-{[(carboxy-2κO)methyl](methyl)amino-2κN}-6-{[(carboxy-3κO)methyl]-(methyl)amino-3κN}cyclohexane-1,3,5-triolate-1κ²O¹,O³:2κ²O³,O⁵:3κ²O¹,O⁵]}trigadolinate(III),
Na₃[Ho₃(H₋₃macita)₂]=Trisodium bis {μ₃-[(all-cis)-2-{[(carboxy-1κO)methyl](methyl)-amino-1κN}-4-{[(carboxy-2κO)methyl](methyl)amino-2κN}-6-{[(carboxy-3κO)methyl]-(methyl)amino-3κN}cyclohexane-1,3,5-triolate-1κ²O¹,O³:2κ²O³,O⁵:3κ²O¹,O⁵]}triholmate (III),
Na₃[Er₃(H₋₃macita)₂]=Trisodium bis {μ₃-[(all-cis)-2-{[(carboxy-1κO)methyl](methyl)-amino-1κN}-4-{[(carboxy-2κO)methyl](methyl)amino-2κN}-6-{[(carboxy-3κO)methyl]-(methyl)amino-3κN}cyclohexane-1,3,5-triolate-1κ²O¹,O³:2κ²O³,O⁵:3κ²O¹,O⁵]}trierbate(III)
Na₃[Yb₃(H₋₃macita)₂]=Trisodium bis {μ₃-[(all-cis)-2-{[(carboxy-1κO)methyl](methyl)-amino-1κN}-4-{[(carboxy-2κO)methyl](methyl)amino-2κN}-6-{[(carboxy-3κO)methyl]-(methyl)amino-3κN}cyclohexane-1,3,5-triolate-1κ²O¹,O³:2κ²O³,O⁵:3κ²O¹,O⁵]}triytterbate(III),
[Hf₃(H₋₃tacitp)₂]=Bis {μ₃-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}trihafnium(IV),
Na₃[Lu₃(H₋₃tacitp)₂]=Trisodium bis {μ₃-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}trilutetate(III)
Na₃[Ho₃(H₋₃tacitp)₂]=Trisodium bis {13-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}triholmate(III),
Na₃[Er₃(H₋₃tacitp)₂]=Trisodium bis {μ₃-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}trierbate(III),
Na₃[Yb₃(H₋₃tacitp)₂]=Trisodium bis {13-[(all-cis)-2-{[(carboxy-1κO)ethyl]amino-1κN}-4-{[(carboxy-2κO)ethyl]amino-2κN}-6-{[(carboxy-3κO)ethyl]amino-3κN}cyclohexane-1,3,5-triolate-1κ²O¹,O³:2κ²O³,O⁵:3κ²O¹,O⁵]}triytterbate(III),
[Hf₃(H₋₃macitp)₂]=Bis {μ₃-[(all-cis)-2-{[(carboxy-1κO)ethyl](methyl)amino-1}-4-{[(carboxy-2κO)ethyl](methyl)amino-2κN}-6-{[(carboxy-3κO)ethyl](methyl)amino-3κN}cyclohexane-1,3,5-triolate-1κ²O¹,O³:2κ²O³,O⁵:3κ²O¹,O⁵]}trihafnium(IV),
Na₃[Lu₃(H₋₃macitp)₂]=Trisodium bis {μ₃-[(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}trilutetate(III),
Na₃[Gd₃(H₋₃macitp)₂]=Trisodium bis {μ₃-[(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}trigadolinate(III),
Na₃[Ho₃(H₋₃macitp)₂]=Trisodium bis {μ₃-[(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}triholmate(III),
Na₃[Er₃(H₋₃macitp)₂]=Trisodium bis {μ₃-[(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}trierbate(III), and
Na₃[Yb₃(H₋₃macitp)₂]=Trisodium bis {μ₃-[(all-cis)-2-{[(carboxy-1κO)ethyl](methyl)-amino-1κN}-4-{[(carboxy-2κO)ethyl](methyl)amino-2κN}-6-{[(carboxy-3κO)ethyl]-(methyl)amino-3κN}cyclohexane-1,3,5-triolate-1κ²O¹,O³:2κ²O³,O⁵:3κ²O¹,O⁵]}triytterbate(III).

8. A process for the preparation of a trinuclear heavy metal complex of the general formula (I) of claim 1 comprising reacting a carboxylic acid of the general formula (II), 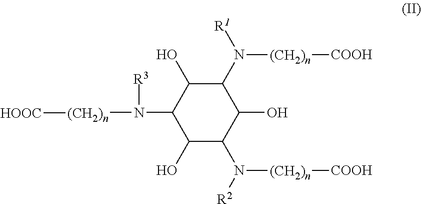
wherein
the substituents at the cyclo hexyl ring exhibit an all-cis configuration;
R¹, R² and R³ are independently H or methyl;
and
n is 1 or 2;
with a metal halogenide,
wherein
metal is Lanthanum, Cerium, Praseodymium, Neodymium, Samarium, Europium, Gadolinium, Terbium, Dysprosium, Holmium, Erbium, Thulium, Ytterbium, Lutetium, Hafnium or Bismuth;
and
halogenide is either chloride or bromide,
and hydrates thereof,
in aqueous solution under elevated temperatures ranging from 80° C. to 160° C. in a pH range of 1 to 6 preferably at 90° to 130° C. in a pH range of 2 to 5.

9. (canceled)

10. Use of the trinuclear metal complex of claim 1, including any protonated species and any deprotonated species of said complex, any isomeric form of said complex, and any pharmaceutically acceptable salt of the complex or hydrate thereof, for the diagnosis of a disease.

11. Use of the trinuclear metal complex of claim 1, including any protonated species and any deprotonated species of said compound, any isomeric form of said complex, and any pharmaceutically acceptable salt of the complex or hydrate thereof, as diagnostic agent, especially X-ray diagnostic agent.

12. (canceled)
